SEQUENCE LISTING (7 sequences)

SEQ ID NO: 1 - DNA, 2088 bp, synthetic construct
  source 1..2088: mol_type = other DNA
atgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactga

SEQ ID NO: 2 - DNA, 1326 bp, synthetic construct
  source 1..1326: mol_type = other DNA
atggcagaacctcgtcaagagtttgaagtcatggaagatcatgctggaacctatggcttgggtgataggaaagatcagggtggatataccatgcaccaagatcaagagggagatactgatgcaggcttgaaagaatctcctttgcagacacctactgaagatggaagtgaggaacctggttctgaaacatcagatgctaagtccacacctacagccgaggatgttaccgctcctttggttgatgaaggtgcaccaggtaaacaagctgctgcacagccacatactgagattccagaaggcaccactgctgaggaggctggcattggcgatacaccaagtttggaggatgaagctgctggacatgttacccaagctagaatggtatctaagtctaaggatggaactggtagtgacgacaagaaggccaaaggagctgacggcaagactaagattgccacacctcgtggagctgcaccaccaggtcagaaaggccaagctaacgctactaggattcctgctaagactcctcctgctcccaagactcctccaagttcaggtgagccaccaaagtcaggtgataggtctggttattcaagtccaggtagtcctggtacaccaggtagtagatccagaactccatctttaccaactcctcctaccagagaaccaaagaaggtagcagtggtaagaactcctcctaagtcaccttcttctgccaagtctaggttacaaaccgcaccagttccaatgccagatttgaagaacgtgaaatctaagattggctcaactgagaacttaaagcaccaaccaggaggtggcaaggttcagatcatcaacaagaagttagatttgtctaatgttcaatcaaagtgtggctctaaggataacatcaaacatgtaccaggaggtggtagtgtacagattgtttacaagcctgtagatttgtctaaagttacctctaagtgtggaagtttgggtaacatccatcataaacctggtggtggccaagtggaggttaagagtgagaaattggactttaaggacagggtacaatcaaagattggcagtttggacaacatcacccacgtgccaggaggtggtaacaagaagattgaaactcacaagttaacctttagagagaatgccaaagctaagactgaccacggagctgagattgtgtacaaatcaccagtcgtatcaggagacacttctccaagacacttatcaaatgtgtcttccactggaagtatagatatggttgactcaccacaattagctaccttggcagatgaagtgtctgcatccttagctaaacaaggtttgtaa

SEQ ID NO: 3 - DNA, 1515 bp, synthetic construct
  source 1..1515: mol_type = other DNA
atgttcccaagagagaagacttggaacatctctttcgctggttgtggattcttaggcgtgtactatgtgggtgtagcaagttgcttgcgtgaacacgctcctttcttagttgccaacgctacacacatctatggagcttccgcaggagctttaactgcaacagccttggtaacaggtgtttgtttaggtgaagcaggtgctaagttcatagaggtttctaaggaagctcgtaagagattcttgggaccattgcacccttcattcaacttagtgaagattatcaggtccttcttattgaaggtattgccagcagattcccacgagcacgctagtggtaggttgggtatttcattaaccagggtttcagatggagagaatgtcatcatatctcatttcaattctaaagacgagttgattcaggccaatgtgtgctctggcttcattcctgtttactgcggtttgattccaccatccttgcaaggtgttagatatgtagatggtggaatatccgataacttacctttgtacgagttgaagaacactattacagtttcacccttctccggtgagtccgacatttgtcctcaggattcttctaccaacattcacgaattgagagtaacaaacacatctatccaattcaatttgaggaatttgtacaggttatccaaggcattgtttcctccagagccattggtcttgagagagatgtgtaagcaaggttatagagatggtttaagattcttgcagcgtaacggcttgttaaacagacccaacccattgttagcattgccacctgccaggccacatggtccagaagataaggatcaagccgtggagtccgcacaagcagaagattactcccaattgccaggagaagaccatgtattagaacacttgcctgcaagattgaatgaagccttattggaagcatgtgtggaacctacagacttattgactactttgagtaacatgttacctgtgagattggccaccgctatgatggttccttacactttaccattggaatccgcattgtcctttactatttgcttattggagtggttaccagatgtgcctgaagacatacgttggatgaaggaacaaacaggttccatttgccagtacttggttatgagggctaaacgtaagttgggtagacacttaccatccagattacctgaacaagtagaattgaggagagttcagtccttgccatctgtccctttatcatgcgcagcttacagagaagctcctcctggttggatgagaaacaatttgtcattgggagacgcattagccaaatgggaagaatgccaacgtcaattgttattgggcttattctgcacaaatgtcgctttccctccagaggctttaagaatgagggctcctgctgatcctgcaccagcaccagccgacccagcctctcctcaacaccaattggcaggtcctgcacccttgttatcaacacctgctcctgaagctagacccgtaattggtgctttgggcttgtaa

SEQ ID NO: 4 - DNA, 1266 bp, synthetic construct
  source 1..1266: mol_type = other DNA
atggcagcaggctttggtagatgttgtagggtattgaggtctatttctaggttccactggagatcacaacacactaaagcaaacaggcagagagaaccaggtttgggtttctcattcgagtttacagaacaacagaaggagttccaagctacagctagaaagtttgctagagaagagataatccctgtcgctgctgaatatgacaagactggtgaatatccagtcccattgattagacgtgcttgggaattaggcttaatgaacacacatattcctgagaattgtggtggcttaggtttgggtactttcgacgcctgcttgatttcagaagagttggcttacggttgcaccggtgttcagactgcaattgaaggtaactcattaggtcagatgccaatcatcatagctggaaatgatcagcagaagaagaagtacttaggaagaatgactgaagaaccattgatgtgcgcttactgcgtcactgaaccaggagctggtagtgacgtagctggaatcaagactaaagcagagaagaaaggtgacgagtacataatcaatggtcagaagatgtggattacaaacggtggtaaggcaaattggtacttcttattggctagatccgacccagatcctaaagctcctgccaataaggctttcacaggtttcattgtagaagccgatacacctggtattcaaataggtcgtaaggaattgaatatgggtcagagatgctccgacaccagaggtatcgtattcgaagacgtgaaagttcccaaagagaatgtgttgattggagatggagcaggattcaaagtagctatgggagccttcgacaagactagaccagtagtagctgctggtgctgtcggtttggctcaaagagccttagatgaagcaactaagtatgctttggagagaaagacattcggtaagttgttggttgagcatcaagctatctctttcatgttggctgagatggccatgaaggttgaattggcaaggatgtcctatcaaagagctgcatgggaggttgattctggcagacgtaatacctattacgctagtattgccaaagccttcgctggagacatcgcaaatcagttagcaactgatgccgtccaaatcttaggtggcaatggtttcaacaccgagtatcctgtagagaagttgatgagagatgctaagatataccagatatacgaaggaaccagtcaaattcaaagattgatcgtagcaagagaacatattgacaagtacaagaattaa

SEQ ID NO: 5 - DNA, 2751 bp, synthetic construct
  source 1..2751: mol_type = other DNA
atggtacagaaggagagtcaagctgcattagaagaaagggaatcagaaaggaatgcaaacccagcatccgttagtggcgcatctttggaaccatctgccgctcctgctccaggtgaagataatccatcaggtgcaggcgctgccgcaggaactggagccgcaggaggagccaggagattcttatgtggtgtcgtagaaggcttctacggcagaccctgggttatggaacagagaaaggagttgttcagaagattgcagaagtgggagttgaatacttacttgtatgctcctaaggatgactacaagcatagaatgttctggcgtgaaatgtattctgtggaagaagctgaacagttgatgacattgatatcagctgcaagagaatacgaaattgagtttatctatgcaatctctccaggcttggacattaccttctccaacccaaaggaggtcagtaccttaaagcgtaagttggatcaagtgtctcaattcggatgtagaagtttcgccttattgttcgatgacatcgaccacaacatgtgcgctgctgacaaagaagtgttctcttctttcgcacatgcacaggtgtccatcacaaatgaaatctatcaatacttaggagaaccagaaacattcttattctgcccaacagagtattgtggcactttctgttatccatccgtcagtcaaagtccctatttgagaactgtgggtgagaagttattgccaggaatcgaagtattgtggactggacctaaagttgtgtctaaagaaattccagtggagtctattgaagaagtatcaaagatcataaagagagcacctgttatctgggataacattcacgctaacgattacgatcagaagagattgttcttgggtccttacaaaggtaggtctaccgagttgattccaaggttgaaaggcgtattgaccaatcctaactgtgaatttgaagcaaactatgtagccattcacacattggctacttggtataagtccaacatgaacggcgtacgtaaagatgttgttatgaccgattccgaagattccacagtatctatccagatcaaattggagaacgaaggtagtgacgaagatatagagactgatgtgttgtactctcctcagatggcattgaaattggccttgactgaatggttgcaggaatttggtgtgccacaccaatactcatcaagacaagtcgctcattccggagctaagacatctgttgtggatggcacacccttggtggcagcaccatctttgaatgcaacaacagttgttactacagtttatcaagaacccattatgtcacagggtgctgctttatccggtgagccctctgctttaactaaagaagaagagaagaaacaacctgatgaggaaccaatggatatggtggttgagaagcaagaagagtctgaacacaagtccgataaccagatcttaacagagattgtcgaagctaagatggcagaagaattgaaacctatggataccgataaagaatctattgccgaaagtaagagtccagaaatgagtatgcaagaagactgtatcaatgatattgcacccatgcagactgatgaacaagccaataaggaacagttcgttccaggcccaaatgagaagcctttgtatgcagcagaacctgtcactttggaagacttgcaattgttagcagatttgttctacttaccatacgagcacggtcctaagggtgcacaaatgttaagagaatttcaatggttaagagctaattcatctgtcgtgtcagtaaactgtaaaggtaaggatagtgagaagattgaagagtggagatcaagagcagccaagttcgaggaaatgtgtgctttagtcatgggcatgtttaccagattgtctaattgcgctaatagaactatcttgtatgatatgtattcatacgtttgggacataaagtccattatgtctatggtcaaatcatttgtacaatggttgggttgtagaagtcactcttctgcccagttcttgattggtgaccaagagccttgggcctttagaggcggattggcaggcgaatttcaacgtttgttacctattgacggtgctaatgatttgttctttcagccaccacctttaactcctacctctaaagtctatactattaggccatactttcccaaagacgaagccagtgtgtataagatctgtagagagatgtacgatgatggtgtaggtttgccatttcaatcccaaccagacttgataggtgacaaattggtaggaggtttgttgtctttatctttagactattgcttcgtgttagaggatgaggatggtatttgcggttatgccttaggaactgtagatgtaactcctttcataaagaaatgcaagatatcttggatacctttcatgcaagagaagtatactaaaccaaatggtgataaggaattgagtgaagctgagaagatcatgttatctttccatgaagaacaagaagtattgccagagacattcttggccaatttcccttctttgataaagatggatatacataagaaggtgactgatccttcagtagccaaatctatgatggcctgcttattgtcatccttaaaggccaatggatcaagaggtgctttctgtgaggtgaggccagatgacaagagaatcttagagttctatagtaagttaggttgtttcgagattgctaagatggaaggtttccctaaggacgtcgtgatcttgggaaggtctttgtaa

SEQ ID NO: 6 - DNA, 717 bp, synthetic construct
  source 1..717: mol_type = other DNA
atgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataa

SEQ ID NO: 7 - DNA, 18972 bp, synthetic construct
  source 1..18972: mol_type = other DNA
acggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatcggactactagcagctgtaatacgactcactatagggaatattaagcttgccaccatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactgagtttaaactcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcgcgccaccatggcagaacctcgtcaagagtttgaagtcatggaagatcatgctggaacctatggcttgggtgataggaaagatcagggtggatataccatgcaccaagatcaagagggagatactgatgcaggcttgaaagaatctcctttgcagacacctactgaagatggaagtgaggaacctggttctgaaacatcagatgctaagtccacacctacagccgaggatgttaccgctcctttggttgatgaaggtgcaccaggtaaacaagctgctgcacagccacatactgagattccagaaggcaccactgctgaggaggctggcattggcgatacaccaagtttggaggatgaagctgctggacatgttacccaagctagaatggtatctaagtctaaggatggaactggtagtgacgacaagaaggccaaaggagctgacggcaagactaagattgccacacctcgtggagctgcaccaccaggtcagaaaggccaagctaacgctactaggattcctgctaagactcctcctgctcccaagactcctccaagttcaggtgagccaccaaagtcaggtgataggtctggttattcaagtccaggtagtcctggtacaccaggtagtagatccagaactccatctttaccaactcctcctaccagagaaccaaagaaggtagcagtggtaagaactcctcctaagtcaccttcttctgccaagtctaggttacaaaccgcaccagttccaatgccagatttgaagaacgtgaaatctaagattggctcaactgagaacttaaagcaccaaccaggaggtggcaaggttcagatcatcaacaagaagttagatttgtctaatgttcaatcaaagtgtggctctaaggataacatcaaacatgtaccaggaggtggtagtgtacagattgtttacaagcctgtagatttgtctaaagttacctctaagtgtggaagtttgggtaacatccatcataaacctggtggtggccaagtggaggttaagagtgagaaattggactttaaggacagggtacaatcaaagattggcagtttggacaacatcacccacgtgccaggaggtggtaacaagaagattgaaactcacaagttaacctttagagagaatgccaaagctaagactgaccacggagctgagattgtgtacaaatcaccagtcgtatcaggagacacttctccaagacacttatcaaatgtgtcttccactggaagtatagatatggttgactcaccacaattagctaccttggcagatgaagtgtctgcatccttagctaaacaaggtttgtaacccgggtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagcttaaggccaccatgttcccaagagagaagacttggaacatctctttcgctggttgtggattcttaggcgtgtactatgtgggtgtagcaagttgcttgcgtgaacacgctcctttcttagttgccaacgctacacacatctatggagcttccgcaggagctttaactgcaacagccttggtaacaggtgtttgtttaggtgaagcaggtgctaagttcatagaggtttctaaggaagctcgtaagagattcttgggaccattgcacccttcattcaacttagtgaagattatcaggtccttcttattgaaggtattgccagcagattcccacgagcacgctagtggtaggttgggtatttcattaaccagggtttcagatggagagaatgtcatcatatctcatttcaattctaaagacgagttgattcaggccaatgtgtgctctggcttcattcctgtttactgcggtttgattccaccatccttgcaaggtgttagatatgtagatggtggaatatccgataacttacctttgtacgagttgaagaacactattacagtttcacccttctccggtgagtccgacatttgtcctcaggattcttctaccaacattcacgaattgagagtaacaaacacatctatccaattcaatttgaggaatttgtacaggttatccaaggcattgtttcctccagagccattggtcttgagagagatgtgtaagcaaggttatagagatggtttaagattcttgcagcgtaacggcttgttaaacagacccaacccattgttagcattgccacctgccaggccacatggtccagaagataaggatcaagccgtggagtccgcacaagcagaagattactcccaattgccaggagaagaccatgtattagaacacttgcctgcaagattgaatgaagccttattggaagcatgtgtggaacctacagacttattgactactttgagtaacatgttacctgtgagattggccaccgctatgatggttccttacactttaccattggaatccgcattgtcctttactatttgcttattggagtggttaccagatgtgcctgaagacatacgttggatgaaggaacaaacaggttccatttgccagtacttggttatgagggctaaacgtaagttgggtagacacttaccatccagattacctgaacaagtagaattgaggagagttcagtccttgccatctgtccctttatcatgcgcagcttacagagaagctcctcctggttggatgagaaacaatttgtcattgggagacgcattagccaaatgggaagaatgccaacgtcaattgttattgggcttattctgcacaaatgtcgctttccctccagaggctttaagaatgagggctcctgctgatcctgcaccagcaccagccgacccagcctctcctcaacaccaattggcaggtcctgcacccttgttatcaacacctgctcctgaagctagacccgtaattggtgctttgggcttgtaagcatgctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggtcgacgccaccatggcagcaggctttggtagatgttgtagggtattgaggtctatttctaggttccactggagatcacaacacactaaagcaaacaggcagagagaaccaggtttgggtttctcattcgagtttacagaacaacagaaggagttccaagctacagctagaaagtttgctagagaagagataatccctgtcgctgctgaatatgacaagactggtgaatatccagtcccattgattagacgtgcttgggaattaggcttaatgaacacacatattcctgagaattgtggtggcttaggtttgggtactttcgacgcctgcttgatttcagaagagttggcttacggttgcaccggtgttcagactgcaattgaaggtaactcattaggtcagatgccaatcatcatagctggaaatgatcagcagaagaagaagtacttaggaagaatgactgaagaaccattgatgtgcgcttactgcgtcactgaaccaggagctggtagtgacgtagctggaatcaagactaaagcagagaagaaaggtgacgagtacataatcaatggtcagaagatgtggattacaaacggtggtaaggcaaattggtacttcttattggctagatccgacccagatcctaaagctcctgccaataaggctttcacaggtttcattgtagaagccgatacacctggtattcaaataggtcgtaaggaattgaatatgggtcagagatgctccgacaccagaggtatcgtattcgaagacgtgaaagttcccaaagagaatgtgttgattggagatggagcaggattcaaagtagctatgggagccttcgacaagactagaccagtagtagctgctggtgctgtcggtttggctcaaagagccttagatgaagcaactaagtatgctttggagagaaagacattcggtaagttgttggttgagcatcaagctatctctttcatgttggctgagatggccatgaaggttgaattggcaaggatgtcctatcaaagagctgcatgggaggttgattctggcagacgtaatacctattacgctagtattgccaaagccttcgctggagacatcgcaaatcagttagcaactgatgccgtccaaatcttaggtggcaatggtttcaacaccgagtatcctgtagagaagttgatgagagatgctaagatataccagatatacgaaggaaccagtcaaattcaaagattgatcgtagcaagagaacatattgacaagtacaagaattaattaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagctcgaggccaccatggtacagaaggagagtcaagctgcattagaagaaagggaatcagaaaggaatgcaaacccagcatccgttagtggcgcatctttggaaccatctgccgctcctgctccaggtgaagataatccatcaggtgcaggcgctgccgcaggaactggagccgcaggaggagccaggagattcttatgtggtgtcgtagaaggcttctacggcagaccctgggttatggaacagagaaaggagttgttcagaagattgcagaagtgggagttgaatacttacttgtatgctcctaaggatgactacaagcatagaatgttctggcgtgaaatgtattctgtggaagaagctgaacagttgatgacattgatatcagctgcaagagaatacgaaattgagtttatctatgcaatctctccaggcttggacattaccttctccaacccaaaggaggtcagtaccttaaagcgtaagttggatcaagtgtctcaattcggatgtagaagtttcgccttattgttcgatgacatcgaccacaacatgtgcgctgctgacaaagaagtgttctcttctttcgcacatgcacaggtgtccatcacaaatgaaatctatcaatacttaggagaaccagaaacattcttattctgcccaacagagtattgtggcactttctgttatccatccgtcagtcaaagtccctatttgagaactgtgggtgagaagttattgccaggaatcgaagtattgtggactggacctaaagttgtgtctaaagaaattccagtggagtctattgaagaagtatcaaagatcataaagagagcacctgttatctgggataacattcacgctaacgattacgatcagaagagattgttcttgggtccttacaaaggtaggtctaccgagttgattccaaggttgaaaggcgtattgaccaatcctaactgtgaatttgaagcaaactatgtagccattcacacattggctacttggtataagtccaacatgaacggcgtacgtaaagatgttgttatgaccgattccgaagattccacagtatctatccagatcaaattggagaacgaaggtagtgacgaagatatagagactgatgtgttgtactctcctcagatggcattgaaattggccttgactgaatggttgcaggaatttggtgtgccacaccaatactcatcaagacaagtcgctcattccggagctaagacatctgttgtggatggcacacccttggtggcagcaccatctttgaatgcaacaacagttgttactacagtttatcaagaacccattatgtcacagggtgctgctttatccggtgagccctctgctttaactaaagaagaagagaagaaacaacctgatgaggaaccaatggatatggtggttgagaagcaagaagagtctgaacacaagtccgataaccagatcttaacagagattgtcgaagctaagatggcagaagaattgaaacctatggataccgataaagaatctattgccgaaagtaagagtccagaaatgagtatgcaagaagactgtatcaatgatattgcacccatgcagactgatgaacaagccaataaggaacagttcgttccaggcccaaatgagaagcctttgtatgcagcagaacctgtcactttggaagacttgcaattgttagcagatttgttctacttaccatacgagcacggtcctaagggtgcacaaatgttaagagaatttcaatggttaagagctaattcatctgtcgtgtcagtaaactgtaaaggtaaggatagtgagaagattgaagagtggagatcaagagcagccaagttcgaggaaatgtgtgctttagtcatgggcatgtttaccagattgtctaattgcgctaatagaactatcttgtatgatatgtattcatacgtttgggacataaagtccattatgtctatggtcaaatcatttgtacaatggttgggttgtagaagtcactcttctgcccagttcttgattggtgaccaagagccttgggcctttagaggcggattggcaggcgaatttcaacgtttgttacctattgacggtgctaatgatttgttctttcagccaccacctttaactcctacctctaaagtctatactattaggccatactttcccaaagacgaagccagtgtgtataagatctgtagagagatgtacgatgatggtgtaggtttgccatttcaatcccaaccagacttgataggtgacaaattggtaggaggtttgttgtctttatctttagactattgcttcgtgttagaggatgaggatggtatttgcggttatgccttaggaactgtagatgtaactcctttcataaagaaatgcaagatatcttggatacctttcatgcaagagaagtatactaaaccaaatggtgataaggaattgagtgaagctgagaagatcatgttatctttccatgaagaacaagaagtattgccagagacattcttggccaatttcccttctttgataaagatggatatacataagaaggtgactgatccttcagtagccaaatctatgatggcctgcttattgtcatccttaaaggccaatggatcaagaggtgctttctgtgaggtgaggccagatgacaagagaatcttagagttctatagtaagttaggttgtttcgagattgctaagatggaaggtttccctaaggacgtcgtgatcttgggaaggtctttgtaagaattctcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgccggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggaggcggccgccatgtctaaaggtgaagaattattcactggtgttgtcccaattttggttgaattagatggtgatgttaatggtcacaaattttctgtctccggtgaaggtgaaggtgatgctacttacggtaaattgaccttaaaatttatttgtactactggtaaattgccagttccatggccaaccttagtcactactttcggttatggtgttcaatgttttgctagatacccagatcatatgaaacaacatgactttttcaagtctgccatgccagaaggttatgttcaagaaagaactatttttttcaaagatgacggtaactacaagaccagagctgaagtcaagtttgaaggtgataccttagttaatagaatcgaattaaaaggtattgattttaaagaagatggtaacattttaggtcacaaattggaatacaactataactctcacaatgtttacatcatggctgacaaacaaaagaatggtatcaaagttaacttcaaaattagacacaacattgaagatggttctgttcaattagctgaccattatcaacaaaatactccaattggtgatggtccagtcttgttaccagacaaccattacttatccactcaatctgccttatccaaagatccaaacgaaaagagagaccacatggtcttgttagaatttgttactgctgctggtattacccatggtatggatgaattgtacaaataatctagagggccgcatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagcgcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttggcattgctacaggcatcgtggtgtcactctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataatagtgtatcacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctagcttatcgatgataagctgtcaaagatgagaattaattccacggactatagactatactagatactccgtctactgtacgatacacttccgctcaggtccttgtcctttaacgaggccttaccactcttttgttactctattgatccagctcagcaaaggcagtgtgatctaagattctatcttcgcgatgtagtaaaactagctagaccgagaaagagactagaaatgcaaaaggcacttctacaatggctgccatcattattatccgatgtgacgctgcagcttctcaatgatattcgaatacgctttgaggagatacagcctaatatccgacaaactgttttacagatttacgatcgtacttgttacccatcattgaattttgaacatccgaacctgggagttttccctgaaacagatagtatatttgaacctgtataataatatatagtctagcgctttacggaagacaatgtatgtatttcggttcctggagaaactattgcatctattgcataggtaatcttgcacgtcgcatccccggttcattttctgcgtttccatcttgcacttcaatagcatatctttgttaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgacgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatgggaagctccaccccggttgataatcagaaaagccccaaaaacaggaagattgtataagcaaatatttaaattgtaaacgttaatattttgttaaaattcgcgttaaatttttgttaaatcagctcattttttaacgaatagcccgaaatcggcaaaatcccttataaatcaaaagaatagaccgagatagggttgagtgttgttccagtttccaacaagagtccactattaaagaacgtggactccaacgtcaaagggcgaaaaagggtctatcagggcgatggcccactacgtgaaccatcaccctaatcaagttttttggggtcgaggtgccgtaaagcagtaaatcggaagggtaaacggatgcccccatttagagcttgacggggaaagccggcgaacgtggcgagaaaggaagggaagaaagcgaaaggagcgggggctagggcggtgggaagtgtaggggtcacgctgggcgtaaccaccacacccgccgcgcttaatggggcgctacagggcgcgtggggatgatccactagt

(19) United States
(12) Patent Application Publication
(10) Pub. No.: US 2025/0231201 A1
(43) Pub. Date: Jul. 17, 2025

(54) BIOLOGICAL DEVICES FOR THE DETECTION OF ALZHEIMER'S DISEASE AND CONCUSSIONS AND METHODS OF USE THEREOF

(71) Applicant: Bio Capital Holdings, LLC, Houston, TX (US)

(72) Inventor: Raul CUERO RENGIFO, Cypress, TX (US)

(21) Appl. No.: 19/013,087

(22) Filed: Jan. 8, 2025

Related U.S. Application Data
(60) Provisional application No. 63/619,819, filed on Jan. 11, 2024.

(51) Int. Cl. G01N 33/68; C07K 14/47; C12N 1/06; C12N 1/18; C12N 9/02; C12N 9/18; C12N 9/24; C12N 15/81; G01N 21/64

(52) U.S. Cl. CPC G01N 33/6896; C07K 14/4711; C12N 1/063; C12N 1/18; C12N 9/001; C12N 9/18; C12N 9/2402; C12N 15/81; G01N 21/6486; C12Y 103/99003; C12Y 301/01003; C12Y 302/01169; G01N 2333/4709; G01N 2333/90206; G01N 2333/918; G01N 2333/924; G01N 2800/2821

(57) ABSTRACT

Described herein are biological devices and extracts useful for detecting Alzheimer's disease and/or concussions. The biological devices include microbial cells transformed with a DNA construct containing genes for producing β-amyloid precursor protein, microtubule associated protein tau, adipose triglyceride lipase, acyl-CoA dehydrogenase, and O-linked N-acetylglucosamine transferase. In some instances, the biological devices also include a gene for enhanced green fluorescent protein. Methods for using the devices to diagnose or detect Alzheimer's disease and/or concussions are also provided herein.

### CROSS-REFERENCE TO RELATED APPLICATIONS

This application claims the benefit of U.S. Provisional Application Ser. No. 63/619,819, filed Jan. 11, 2024, which is incorporated herein by reference in its entirety.

### CROSS REFERENCE TO SEQUENCE LISTING

The genetic components described herein are referred to by sequence identifier numbers (SEQ ID NO). The SEQ ID NOs correspond numerically to the sequence identifiers <400>1, <400>2, etc. The sequence listing in written computer readable format (CRF) as a text file named “930201-8130_Sequence_Listing.xml” created on Dec. 11, 2023, and having a size of 35,603 bytes, is incorporated by reference in its entirety.

### BACKGROUND

The term “dementia” describes a set of symptoms that can include loss of memory, mood changes, and problems with communication and reasoning. Alzheimer's disease is the most common form of dementia and affects more than 26 million people worldwide. Incidence of Alzheimer's disease is expected to increase as the world's population ages; currently, there is no cure for this disease. In some cases, traumatic brain injuries (TBIs) such as concussion may be associated with an increased dementia risk later in life.

The causes of Alzheimer's disease are not well-understood, though abnormal structure called plaques and tangles have been identified in the brains of deceased Alzheimer's patients. Plaques are deposits of a protein fragment called β-amyloid, whereas tangles are twisted fibers of a different protein (tau) that accumulates inside cells. TBIs have been found, in some cases, to be associated with an immediate rise misfolded β-amyloid.

Due to the difficulty of accessing cerebral tissue in a living organism, progress in the study and understanding of Alzheimer's disease and other conditions associated with β-amyloid deposits and/or tau tangles has been slow. Additionally, a definitive diagnosis of Alzheimer's disease can only be made through autopsy, after death. Therefore, a need exists for earlier diagnosis of Alzheimer's disease, even before the appearance of clinical symptoms, so that intervention can be administered to slow the progress of the disease or to mitigate Alzheimer's symptoms. It would further be desirable if the same system could be applied to the assessment of individuals having concussions or other TBIs. These needs and other needs are satisfied by the present disclosure.

### SUMMARY

Described herein are biological devices and extracts useful for detecting Alzheimer's disease and/or concussions. The biological devices include microbial cells transformed with a DNA construct containing genes for producing β-amyloid precursor protein, microtubule associated protein tau, adipose triglyceride lipase, acyl-CoA dehydrogenase, and O-linked N-acetylglucosamine transferase. In some instances, the biological devices also include a gene for enhanced green fluorescent protein. Methods for using the devices to diagnose or detect Alzheimer's disease and/or concussions are also provided herein.

The advantages of the invention will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

Many aspects of the present disclosure can be better understood with reference to the following drawings, which are incorporated in and constitute a part of this specification. The components in the drawings are not necessarily to scale, emphasis instead being placed upon clearly illustrating the principles of the present disclosure.

FIGS. 1A-1B show, respectively, linear and circular maps of an exemplary biological device.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein are DNA constructs containing the following genetic components:
(a) a gene that encodes a β-amyloid precursor protein;
(b) a gene that encodes microtubule associated protein tau (MAPT);
(c) a gene that encodes adipose triglyceride lipase;
(d) a gene that encodes an acyl-CoA dehydrogenase; and
(e) a gene that encodes an O-linked N-acetylglucosamine transferase (OGlcNAcase).

The DNA constructs may variously encode genes encoding reporter proteins, genes encoding resistance to one or more antibiotics, and the like, and may include regulatory sequences including promoters, terminators, ribosomal binding sites, LAC operons, or other components necessary for the replication of and expression of the genes encoded by the DNA constructs inside microbial hosts such as, for example, Saccharomyces cerevisiae, Escherichia coli, and other microorganisms. Also disclosed are vectors including the DNA constructs and biological devices consisting of host cells that include one or more copies of the vectors.

Also disclosed herein are methods for producing a composition useful for the detection of Alzheimer's disease and/or concussion, the methods including at least the step of culturing the biological devices for a period of time sufficient to produce the composition. Exemplary methods for producing the compositions are disclosed in the Examples.

Further disclosed herein are non-invasive methods for detecting Alzheimer's disease and/or concussion in a subject using the disclosed devices and compositions. In one aspect, disclosed herein is method for detecting β-amyloid protein or concussion in a subject, the method including at least the step of admixing a disclosed composition with a biological sample from the subject and detecting a signal from the biological sample. In an aspect, the subject is a human. In another aspect, the biological can be comprises blood, serum, plasma, or saliva and the signal can be a fluorescence signal.

Before the present compounds, compositions, articles, devices, and/or methods are disclosed and described, it is to be understood that the aspects described below are not limited to specific compounds, synthetic methods, or uses, as such may, of course, vary. It is also to be understood that the terminology used herein is for the purpose of describing particular aspects only and is not intended to be limiting.

In this specification and in the claims that follow, reference will be made to a number of terms that shall defined to have the following meanings:

It must be noted that, as used in the specification and the appended claims, the singular forms “a,” “an,” and “the” include plural referents unless the context clearly dictates otherwise. Thus, for example, reference to “a metabolite” includes mixtures of two or more such metabolites, and the like.

“Optional” or “optionally” means that the subsequently described event or circumstance can or cannot occur, and that the description includes instances where the event or circumstance occurs and instances where it does not. For example, the phrase “a microorganism is optionally genetically modified” means that the microorganism may or may not be genetically modified.

Throughout this specification, unless the context dictates otherwise, the word “comprise,” or variations such as “comprises” or “comprising,” will be understood to imply the inclusion of a stated element, integer, step, or group of elements, integers, or steps, but not the exclusion of any other element, integer, step, or group of elements, integers, or steps.

As used herein, the term “about” is used to provide flexibility to a numerical range endpoint by providing that a given numerical value may be “a little above” or “a little below” the endpoint without affecting the desired result. For purposes of the parent disclosure, “about” refers to a range extending from 10% below the numerical value to 10% above the numerical value. For example, if the numerical value is 10, “about 10” means between 9 and 11, inclusive of the endpoints 9 and 11.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’ The range can also be expressed as an upper limit, e.g. ‘about x, y, z, or less’ and should be interpreted to include the specific ranges of ‘about x,’ ‘about y’, and ‘about z’ as well as the ranges of ‘less than x’, less than y′, and ‘less than z’. Likewise, the phrase ‘about x, y, z, or greater’ should be interpreted to include the specific ranges of ‘about x,’ ‘about y,’ and ‘about z’ as well as the ranges of ‘greater than x,’ greater than y,′ and ‘greater than z.’ In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

Disclosed are materials and components that can be used for, can be used in conjunction with, can be used in preparation for, or are products of the disclosed compositions and methods. These and other materials are disclosed herein, and it is understood that when combinations, subsets, interactions, groups, etc., of these materials are disclosed that while specific reference to each various individual and collective combination and permutation of these compounds may not be explicitly disclosed, each is specifically contemplated and described herein. For example, if an O-linked N-acetylglucosamine transferase is disclosed and discussed and a number of different proteins that can be glycosylated with N-acetylglucosamine are discussed, each and every combination and permutation of O-linked N-acetylglucosamine transferase and protein that is possible is specifically contemplated unless specifically indicated to the contrary. For example, if a class of molecules A, B, and C are disclosed as well as a class of molecules D, E, and F, and an example of a combination molecule, A-D, is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the subgroup of A-E, B-F, and C-E is specifically contemplated and should be considered disclosed from disclosure of A, B, and C; D, E, and F; and the example combination A-D. This concept applies to all aspects of this disclosure including, but not limited to, steps in methods of making and using the disclosed compositions. Thus, if there are a variety of additional steps that can be performed, it is understood that each of these additional steps can be performed with any specific embodiment or combination of elements of the disclosed methods, and that each such combination is specifically contemplated and should be considered disclosed.

References in the specification and concluding claims to parts by weight, of a particular element or component in a composition or article, denote the weight relationship between the element or component and any other elements or components in the composition or article for which a part by weight is expressed. Thus, in a composition containing 2 parts by weight of component X and 5 parts by weight of component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

### DNA Constructs and Biological Devices

In one aspect, cells transformed with a DNA construct can be used in the methods described herein. It is understood that one way to define the variants and derivatives of the genetic components and DNA constructs described herein is in terms of homology/identity to specific known sequences. Those of skill in the art readily understand how to determine the homology of two nucleic acids. For example, the homology can be calculated after aligning two sequences so that the homology is at its highest level. Another way of calculating homology can be performed according to published algorithms (see Zuker, M., Science, 244:48-52, 1989; Jaeger et al, Proc. Natl. Acad. Sci. USA, 86:7706-7710, 1989; Jaeger et al, Methods Enzymol., 183:281-306, 1989, which are herein incorporated by reference for at least material related to nucleic acid alignment).

As used herein, “conservative” mutations are mutations that result in an amino acid change in the protein produced from a sequence of DNA. When a conservative mutation occurs, the new amino acid has similar properties as the wild type amino acid and generally does not drastically change the function or folding of the protein (e.g., switching isoleucine for valine is a conservative mutation since both are small, branched, hydrophobic amino acids). “Silent mutations,” meanwhile, change the nucleic acid sequence of a gene encoding a protein but do not change the amino acid sequence of the protein.

It is understood that the description of mutations and homology can be combined together in any combination, such as embodiments that have at least about 70%, about 75%, about 80%, about 85%, about 90%, about 95%, or about 99% homology to a particular sequence wherein the variants are conservative or silent mutations. It is understood that any of the sequences described herein can be a variant or derivative having the homology values listed above.

In some aspects, genes of interest can be spliced into suitable vectors using restriction enzymes and/or other techniques known in the art. Further in this aspect, synthesis and/or isolation of the genes of interest prior to inclusion in the disclosed constructs may result in the addition of C-terminal and/or N-terminal sequence data including, but not limited to, restriction enzyme recognition sites, linking bases, short segments of chromosomal DNA (including introns or portions of introns if the sequences originate from eukaryotic cells), transposons, nucleotide repeats, regulatory sequences, and/or other material that do not contribute to the known structure of the expressed protein, or are not part of the expressed protein's active site. In one aspect, presence of these remnants may lead to somewhat reduced homology with respect to gene sequence, but the DNA constructs encoding the same can still produce proteins having the desired sequence, active site, and function.

In another aspect, many eukaryotic genes include introns and mRNAs produced during transcription of the same can be spliced differently, producing several transcript variants from the same gene but having slightly different sequences (i.e., reduced levels of homology). In one aspect, different transcript variants can produce proteins having the same active site but differing in another way (e.g. in C-terminal or N-terminal sequence, affecting assembly of protein subunits or other folding processes, cellular localization of the peptides or proteins, or activity level of the peptides or proteins produced due to differential regulation, or the like.

In one aspect, a database such as, for example, GenBank, can be used to determine the sequences of genes and/or regulatory regions of interest, the species from which these elements originate, and related homologous sequences.

In one aspect, the nucleic acids used in the DNA constructs described herein can be amplified using polymerase chain reaction (PCR) prior to being ligated into a plasmid or other vector. Typically, PCR-amplification techniques make use of primers, or short, chemically-synthesized oligonucleotides that are complementary to regions on each respective strand flanking the DNA or nucleotide sequence to be amplified. A person having ordinary skill in the art will be able to design or choose primers based on the desired experimental conditions. In general, primers should be designed to provide for both efficient and faithful replication of the target nucleic acids. Two primers are required for the amplification of each gene, one for the sense strand (that is, the strand containing the gene of interest) and one for the antisense strand (that is, the strand complementary to the gene of interest). Pairs of primers should have similar melting temperatures that are close to the PCR reaction's annealing temperature. In order to facilitate the PCR reaction, the following features should be avoided in primers: mononucleotide repeats, complementarity with other primers in the mixture, self-complementarity, and internal hairpins and/or loops. Methods of primer design are known in the art; additionally, computer programs exist that can assist the skilled practitioner with primer design. Primers can optionally incorporate restriction enzyme recognition sites at their 5′ ends to assist in later ligation into plasmids or other vectors.

PCR can be carried out using purified DNA, unpurified DNA that is integrated into a vector, or unpurified genomic DNA. The process for amplifying target DNA using PCR consists of introducing an excess of two primers having the characteristics described above to a mixture containing the sequence to be amplified, followed by a series of thermal cycles in the presence of a heat-tolerant or thermophilic DNA polymerase, such as, for example, any of Taq, Pfu, Pwo, Tfl, rTth, Tli, or Tma polymerases. A PCR “cycle” involves denaturation of the DNA through heating, followed by annealing of the primers to the target DNA, followed by extension of the primers using the thermophilic DNA polymerase and a supply of deoxynucleotide triphosphates (i.e., dCTP, dATP, dGTP, and TTP), along with buffers, salts, and other reagents as needed. In one aspect, the DNA segments created by primer extension during the PCR process can serve as templates for additional PCR cycles. Many PCR cycles can be performed to generate a large concentration of target DNA or genes. PCR can optionally be performed in a device or machine with programmable temperature cycles for denaturation, annealing, and extension steps. Further, PCR can be performed on multiple genes simultaneously in the same reaction vessel or microcentrifuge tube since the primers chosen will be specific to selected genes. PCR products can be purified by techniques known in the art such as, for example, gel electrophoresis followed by extraction from the gel using commercial kits and reagents.

In a further aspect, the plasmid can include an origin of replication, allowing it to use the host cell's replication machinery to create copies of itself.

As used herein, “operably linked” refers to the association of nucleic acid sequences on a single nucleic acid fragment so that the function of one affects the function of another. For example, if sequences for multiple genes are inserted into a single plasmid, their expression may be operably linked. Alternatively, a promoter is said to be operably linked with a coding sequence when it is capable of affecting the expression of that coding sequence.

As used herein, “expression” refers to transcription and/or accumulation of an mRNA derived from a gene or DNA fragment. Expression may also be used to refer to translation of mRNA into a peptide, polypeptide, or protein.

DNA Constructs for the Detection of Alzheimer's Disease and/or Concussions

In one aspect, provided herein are DNA constructs having at least the following genetic components:
(a) a gene that encodes a β-amyloid precursor protein;
(b) a gene that encodes MAPT;
(c) a gene that encodes adipose triglyceride lipase;
(d) a gene that encodes an acyl CoA dehydrogenase; and
(e) a gene that encodes an OGlcNAcase.

Each component of the DNA constructs is described in detail below.

In one aspect, the DNA constructs disclosed herein incorporate a gene that encodes amyloid precursor protein. In a further aspect, amyloid precursor protein is an integral membrane protein that acts as a cell surface receptor and may be involved with regulation of neuron and synapse development, iron export, and other functions. Amyloid precursor protein undergoes extensive post-translational modification including, but not limited to, sulfation, glycosylation, phosphorylation, sialylation, and proteolysis. Amyloid precursor protein is of interest to the study of Alzheimer's disease since individuals diagnosed with Alzheimer's disease display a great deal of diversity in this gene due to somatic recombination in the brain.

In one aspect, the gene that encodes amyloid precursor protein (also referred to amyloid β precursor protein and/or β amyloid precursor protein) is isolated from a mammal such as, for example, human, chimpanzee, Western lowland gorilla, bonobo, Bornean orangutan, Northern white-cheeked gibbon, Sumatran orangutan, siamang, silvery gibbon, olive baboon, golden snub-nosed monkey, gelada, crab-eating macaque, Indochinese rhesus macaque, sooty mangabey, Tibetan macaque, black-and-white snub-nosed monkey, Francois' langur, Southern pig-tailed macaque, Angola colobus, green monkey, Ugandan red colobus, drill, tufted capuchin, Panamanian white-faced capuchin, common marmoset, black-capped squirrel monkey, gray mouse lemur, Przewalski's horse, horse, Philippine tarsier, plains zebra, African wild ass, East African hippopotamus, Blainville's beaked whale, Sunda slow loris, Alpine marmot, groundhog, Sunda pangolin, South-central black rhinoceros, beluga whale, Chinese pangolin, yellow-bellied marmot, Rice's whale, Eastern gray squirrel, short-beaked common dolphin, Northern treeshrew, blue whale, narrow-ridged finless porpoise, common minke whale, vaquita, Pacific white-sided dolphin, Arctic ground squirrel, large flying fox, common warthog, Indian flying fox, baiji, black flying fox, common bottlenose dolphin, long-finned pilot whale, orca, wild Bactrian camel, dromedary camel, alpaca, Chinese rufous horseshoe bat, wild boar, American black bear, cape golden mole, naked mole-rat, Hawaiian monk seal, polar bear, greater horseshoe bat, Parnell's mustached bat, cape elephant shrew, or brown bear In a further aspect, the gene that encodes amyloid precursor protein has SEQ ID NO. 1 or at least 70% homology thereto, at least 75% homology thereto, at least 80% homology thereto, at least 85% homology thereto, at least 90% homology thereto, at least 95% homology thereto, or at least 99% homology thereto.

Other sequences encoding amyloid precursor protein or related or homologous genes can be identified in a database such as, for example, GenBank. In one aspect, the gene that encodes amyloid precursor protein is isolated from Homo sapiens and can be identified by the GI number NM 201414.3 in the GenBank database. In another aspect, sequences useful herein include those with GI numbers listed in Table 1:

TABLE 1
Amyloid β Precursor Protein

| Source Organism | Sequence Description | GI Number |
|---|---|---|
| Homo sapiens | Amyloid β precursor protein | NM_201414.3 |
| synthetic construct | Amyloid β precursor protein | EU716635.1 |
| Homo sapiens | Amyloid β precursor protein | Y00264.1 |
| Pan troglodytes | Amyloid β precursor protein | XM_009452766.4 |
| Gorilla gorilla | Amyloid β precursor protein | XM_004062635.4 |
| gorilla | | |
| Pan paniscus | Amyloid β precursor protein | XM_008969776.5 |
| Pongo pygmaeus | Amyloid β precursor protein | XM_054468710.1 |
| Homo sapiens | Amyloid β precursor protein | NM_001136131.3 |
| Nomascus leucogenys | Amyloid β precursor protein | XM_030806169.1 |
| Homo sapiens | Amyloid β precursor protein | AK296229.1 |
| Pongo abelii | Amyloid β precursor protein | NM_001133542.1 |
| Symphalangus syndactylus | Amyloid β precursor protein | XM_055279353.1 |
| Pan paniscus | Amyloid β precursor protein | XM_055105202.1 |
| Hylobates moloch | Amyloid β precursor protein | XM_032174827.2 |
| Papio anubis | Amyloid β precursor protein | XM_017956498.2 |
| Rhinopithecus roxellana | Amyloid β precursor protein | XM_010383440.2 |
| Theropithecus gelada | Amyloid β precursor protein | XM_025378890.1 |
| Macaca fascicularis | Amyloid β precursor protein | XM_005548887.3 |
| Homo sapiens | Amyloid β precursor protein | AK298861.1 |
| Macaca mulatta | Amyloid β precursor protein | XM_015133072.2 |
| Cercocebus atys | Amyloid β precursor protein | XM_012029848.1 |
| Macaca thibetana | Amyloid β precursor protein | XM_050784746.1 |
| thibetana | | |
| Rhinopithecus bieti | Amyloid β precursor protein | XM_017856490.1 |
| Macaca fascicularis | Amyloid β precursor protein | M58727.1 |
| Trachypithecus francoisi | Amyloid β precursor protein | XM_033232228.1 |
| Macaca nemestrina | Amyloid β precursor protein | XM_011726340.2 |
| Colobus angolensis | Amyloid β precursor protein | XM_011926494.1 |
| palliatus | | |
| Chlorocebus sabaeus | Amyloid β precursor protein | XM_007966074.2 |
| Homo sapiens | Amyloid β precursor protein | NM_001204303.2 |
| Piliocolobus tephrosceles | Amyloid β precursor protein | XM_023190025.1 |
| Pan troglodytes | Amyloid β precursor protein | XM_009453143.4 |
| Gorilla gorilla gorilla | Amyloid β precursor protein | XM_004062638.4 |
| Mandrillus leucophaeus | Amyloid β precursor protein | XM_011998311.1 |
| Theropithecus gelada | Amyloid β precursor protein | XM_025378892.1 |
| Colobus angolensis | Amyloid β precursor protein | XM_011926496.1 |
| palliatus | | |
| Pongo pygmaeus | Amyloid β precursor protein | XM_054468711.1 |
| Sapajus apella | Amyloid β precursor protein | XM_032277110.1 |
| Cebus imitator | Amyloid β precursor protein | XM_017541908.1 |
| Callithrix jacchus | Amyloid β precursor protein | XM_008986526.4 |
| Saimiri boliviensis | Amyloid β precursor protein | XM_039480481.1 |
| boliviensis | | |
| Sapajus apella | Amyloid β precursor protein | XM_032277112.1 |
| Cebus imitator | Amyloid β precursor protein | XM_037743245.1 |
| Cebus imitator | Amyloid β precursor protein | XM_037743240.1 |
| Homo sapiens | Amyloid β precursor protein | NM_001136129.3 |
| Homo sapiens | Amyloid β precursor protein | AF282245.1 |
| Microcebus murinus | Amyloid β precursor protein | XM_012764455.1 |
| Theropithecus gelada | Amyloid β precursor protein | XM_025378893.1 |
| Colobus angolensis | Amyloid β precursor protein | XM_011926497.1 |
| palliatus | | |
| Equus przewalskii | Amyloid β precursor protein | XM_008514150.1 |
| Equus caballus | Amyloid β precursor protein | XM_003364171.4 |
| Carlito syrichta | Amyloid β precursor protein | XM_008049942.2 |
| Equus quagga | Amyloid β precursor protein | XM_046648235.1 |
| Equus asinus | Amyloid β precursor protein | XM_014859018.2 |
| Pan troglodytes | Amyloid β precursor protein | AK304995.1 |
| Hippopotamus amphibius | Amyloid β precursor protein | XM_057696997.1 |
| kiboko | | |
| Mesoplodon densirostris | Amyloid β precursor protein | XM_060098598.1 |
| Nycticebus coucang | Amyloid β precursor protein | XM_053565416.1 |
| Marmota marmota | Amyloid β precursor protein | XM_048803537.1 |
| marmota | | |
| Marmota monax | Amyloid β precursor protein | XM_046430453.2 |
| Manis javanica | Amyloid β precursor protein | XM_017650814.2 |
| Diceros bicornis minor | Amyloid β precursor protein | XM_058522959.1 |
| Delphinapterus leucas | Amyloid β precursor protein | XM_030760806.1 |
| Manis pentadactyla | Amyloid β precursor protein | XM_036875244.2 |
| Marmota flaviventris | Amyloid β precursor protein | XM_027929080.2 |
| Balaenoptera ricei | Amyloid β precursor protein | XM_059921406.1 |
| Sciurus carolinensis | Amyloid β precursor protein | XM_047564663.1 |
| Delphinus delphis | Amyloid β precursor protein | XM_060010439.1 |
| Tupaia belangeri | Amyloid β precursor protein | KF479228.2 |
| Balaenoptera musculus | Amyloid β precursor protein | XM_036850817.1 |
| Neophocaena asiaeorientalis | Amyloid β precursor protein | XM_024732019.1 |
| asiaeorientalis | | |
| Tupaia belangeri | Amyloid β precursor protein | KY399770.1 |
| Balaenoptera acutorostrata | Amyloid β precursor protein | XM_057545416.1 |
| Phocoena sinus | Amyloid β precursor protein | XM_032629784.1 |
| Lagenorhynchus obliquidens | Amyloid β precursor protein | XM_027101902.1 |
| Urocitellus parryii | Amyloid β precursor protein | XM_026396150.1 |
| Pteropus vampyrus | Amyloid β precursor protein | XM_011358211.2 |
| Phacochoerus africanus | Amyloid β precursor protein | XM_047795061.1 |
| Pteropus giganteus | Amyloid β precursor protein | XM_039858091.1 |
| Lipotes vexillifer | Amyloid β precursor protein | XM_007466496.1 |
| Pteropus alecto | Amyloid β precursor protein | XM_015586510.2 |
| Tursiops truncatus | Amyloid β precursor protein | XM_033855991.1 |
| Globicephala melas | Amyloid β precursor protein | XM_030880956.2 |
| Orcinus orca | Amyloid β precursor protein | XM_004264482.3 |
| Camelus ferus | Amyloid β precursor protein | XM_032476923.1 |
| Camelus dromedarius | Amyloid β precursor protein | XM_031458292.1 |
| Vicugna pacos | Amyloid β precursor protein | XM_006215213.3 |
| Rhinolophus sinicus | Amyloid β precursor protein | XM_019729868.1 |
| Sus scrofa | Amyloid β precursor protein | AK392671.1 |
| Sus scrofa | Amyloid β precursor protein | XM_005670305.3 |
| Sus scrofa | Amyloid precursor protein | DQ267684.1 |
| | variant 1 | |
| Nycticebus coucang | Amyloid β precursor protein | XM_053565419.1 |
| Ursus americanus | Amyloid β precursor protein | XM_045785474.1 |
| Chrysochloris asiatica | Amyloid β precursor protein | XM_006872473.1 |
| Heterocephalus glaber | Amyloid β precursor protein | XM_004842236.3 |
| Neomonachus schauinslandi | Amyloid β precursor protein | XM_021678096.2 |
| Ursus maritimus | Amyloid β precursor protein | XM_040621080.1 |
| Rhinolophus ferrumequinum | Amyloid β precursor protein | XM_033127076.1 |
| Pteronotus parnellii | Amyloid β precursor protein | XM_054579534.1 |
| mesoamericanus | | |
| Elephantulus edwardii | Amyloid β precursor protein | XM_006896033.1 |
| Ursus arctos | Amyloid β precursor protein | XM_026518287.4 |

In one aspect, the DNA constructs disclosed herein incorporate a gene that encodes microtubule associated protein tau (MAPT). In a further aspect, MAPT is a protein found in neurons that helps stabilize the cytoskeleton thereof. In Individuals with Alzheimer's disease, abnormal forms of MAPT cling to other tau proteins and form tangles; accumulations of tau tangles are believed to be important to the decline in cognitive function associated with Alzheimer's disease. In one aspect, MAPT (also referred to herein as “tau” protein) may be active in both Alzheimer's disease processes and concussion-related processes. Further in this aspect, the tau protein is associated with Alzheimer's pathogenicity as described previously. In another aspect, tau- and/or amyloid-related pathogenicity may not be active initially in the case of concussion, but concussion-induced injuries may lead to pathogenicity related to tau proteins and/or amyloid proteins over time.

In one aspect, the gene that encodes MAPT is isolated from a mammal such as, for example, chimpanzee, human, Northern white-cheeked gibbon, silvery gibbon, Western lowland gorilla, siamang, crab-eating macaque, Bornean orangutan, olive baboon, Southern pig-tailed macaque, drill, Tibetan macaque, green monkey, Indochinese rhesus macaque, gelada, sooty mangabey, Ugandan red colobus, black-and-white snub-nosed monkey, Francois' langur, golden snub-nosed monkey, Northern greater galago, Sunda slow loris, greater horseshoe bat, Coquerel's sifaka, tufted capuchin, Nancy Ma's night monkey, Gairdner's shrewmouse, Panamanian white-faced capuchin, ring-tailed lemur, common marmoset, polar bear, black-capped squirrel monkey, Egyptian fruit bat, Chinese rufous horseshoe bat, green spear-nosed bat, Pacific walrus, Sunda pangolin, gray mouse lemur, brown bear, common vampire bat, white-footed mouse, California sea lion, naked mole rat, house mouse, Middle East blind mole rat, Parnell's mustached bat, grey seal, natal long-fingered bat, Ryukyu mouse, banner-tailed kangaroo rat, harbor seal, striped hyena, Southern grasshopper mouse, Eastern deer mouse, American mink, Arctic fox, European mink, Ord's kangaroo rat, Southern white rhinoceros, Weddell seal, Spanish mole, African grass rat, dingo, dog, stoat, black-footed ferret, ferret, yellow-bellied marmot, bank vole, brown rat, European badger, Damara mole rat, groundhog, prairie vole, jaguar, leopard, creeping vole, common raccoon dog, Indian elephant, plains zebra, wild boar, North American river otter, Syrian hamster, Alpine marmot, Canada lynx, clouded leopard, cheetah, snow leopard, or Geoffroy's cat. In a further aspect, the gene that encodes MAPT has SEQ ID NO. 2 or at least 70% homology thereto, at least 75% homology thereto, at least 80% homology thereto, at least 85% homology thereto, at least 90% homology thereto, at least 95% homology thereto, or at least 99% homology thereto.

Other sequences encoding MAPT or related or homologous genes can be identified in a database such as, for example, GenBank. In one aspect, the gene that encodes MAPT is isolated from Pan troglodytes and can be identified by the GI number XM_009431912.4 in the GenBank database. In another aspect, sequences useful herein include those with GI numbers listed in Table 2:

TABLE 2
Microtubule Associated Protein Tau

| Source Organism | Sequence Description | GI Number |
|---|---|---|
| Pan troglodytes | Microtubule associated | XM_009431912.4 |
| | protein tau | |
| Homo sapiens | Microtubule associated | NM_005910.6 |
| | protein tau | |
| Nomascus leucogenys | Microtubule associated | XM_030799947.1 |
| | protein tau | |
| Hylobates moloch | Microtubule associated | XM_032163959.2 |
| | protein tau | |
| Gorilla gorilla | Microtubule associated | XM_055387816.1 |
| gorilla | protein tau | |
| Symphalangus syndactylus | Microtubule associated | XM_055257660.1 |
| | protein tau | |
| Macaca fascicularis | Microtubule associated | XM_005584540.2 |
| | protein tau | |
| Pongo pygmaeus | Microtubule associated | XM_054458842.1 |
| | protein tau | |
| Papio anubis | Microtubule associated | XM_009190917.3 |
| | protein tau | |
| Macaca nemestrina | Microtubule associated | XM_011718654.2 |
| | protein tau | |
| Mandrillus leucophaeus | Microtubule associated | XM_012001941.1 |
| | protein tau | |
| Macaca thibetana | Microtubule associated | XM_050763701.1 |
| thibetana | protein tau | |
| Chlorocebus sabaeus | Microtubule associated | XM_008012258.2 |
| | protein tau | |
| Macaca mulatta | Microtubule associated | XM_015119954.2 |
| | protein tau | |
| Theropithecus gelada | Microtubule associated | XM_025361787.1 |
| | protein tau | |
| Cercocebus atys | Microtubule associated | XM_012060129.1 |
| | protein tau | |
| Cercocebus atys | Microtubule associated | XM_012060121.1 |
| | protein tau | |
| Piliocolobus | Microtubule associated | XM_023196176.2 |
| tephrosceles | protein tau | |
| Rhinopithecus bieti | Microtubule associated | XM_017871635.1 |
| | protein tau | |
| Trachypithecus | Microtubule associated | XM_033219370.1 |
| francoisi | protein tau | |
| Rhinopithecus | Microtubule associated | XM_010389025.2 |
| roxellana | protein tau | |
| Otolemur garnettii | Microtubule associated | XM_012802935.2 |
| | protein tau | |
| Nycticebus coucang | Microtubule associated | XM_053567814.1 |
| | protein tau | |
| Rhinolophus | Microtubule associated | XM_033089295.1 |
| ferrumequinum | protein tau | |
| Propithecus | Microtubule associated | XM_012655247.1 |
| coquereli | protein tau | |
| Sapajus apella | Microtubule associated | XM_032265993.1 |
| | protein tau | |
| Aotus nancymaae | Microtubule associated | XM_021673726.1 |
| | protein tau | |
| Mus pahari | Microtubule associated | XM_021212151.1 |
| | protein tau | |
| Cebus imitator | Microtubule associated | XM_017549295.2 |
| | protein tau | |
| Lemur catta | Microtubule associated | XM_045525914.1 |
| | protein tau | |
| Callithrix jacchus | Microtubule associated | XM_035300265.2 |
| | protein tau | |
| Callithrix jacchus | Microtubule associated | MK630008.1 |
| | protein tau | |
| Ursus maritimus | Microtubule associated | XM_040640445.1 |
| | protein tau | |
| Saimiri boliviensis | Microtubule associated | XM_010330266.2 |
| boliviensis | protein tau | |
| Rousettus aegyptiacus | Microtubule associated | XM_016139836.2 |
| | protein tau | |
| Rhinolophus sinicus | Microtubule associated | XM_019732959.1 |
| | protein tau | |
| Phyllostomus hastatus | Microtubule associated | XM_045836032.1 |
| | protein tau | |
| Odobenus rosmarus | Microtubule associated | XM_004411094.1 |
| divergens | protein tau | |
| Manis javanica | Microtubule associated | XM_036997258.1 |
| | protein tau | |
| Microcebus murinus | Microtubule associated | XM_012751058.2 |
| | protein tau | |
| Ursus arctos | Microtubule associated | XM_026512571.4 |
| | protein tau | |
| Desmodus rotundus | Microtubule associated | XM_045182198.2 |
| | protein tau | |
| Peromyscus leucopus | Microtubule associated | XM_028882676.2 |
| | protein tau | |
| Zalophus californianus | Microtubule associated | XM_027626386.2 |
| | protein tau | |
| Heterocephalus glaber | Microtubule associated | XM_021247548.1 |
| | protein tau | |
| Mus musculus | Microtubule associated | NM_001038609.3 |
| | protein tau | |
| Mus musculus | Microtubule associated | U12914.1 |
| | protein tau | |
| Mus musculus | Microtubule associated | AK147636.1 |
| | protein tau | |
| Nannospalax galili | Microtubule associated | XM_008821902.3 |
| | protein tau | |
| Pteronotus parnellii | Microtubule associated | XM_054574886.1 |
| mesoamericanus | protein tau | |
| Halichoerus grypus | Microtubule associated | XM_036103112.1 |
| | protein tau | |
| Phyllostomus | Microtubule associated | XM_028520563.2 |
| discolor | protein tau | |
| Phyllostomus | Microtubule associated | XM_036033569.1 |
| discolor | protein tau | |
| Vulpes vulpes | Microtubule associated | XM_025999511.1 |
| | protein tau | |
| Miniopterus natalensis | Microtubule associated | XM_016204952.1 |
| | protein tau | |
| Mus caroli | Microtubule associated | XM_021176411.2 |
| | protein tau | |
| Dipodomys spectabilis | Microtubule associated | XM_042674103.1 |
| | protein tau | |
| Phoca vitulina | Microtubule associated | XM_032429420.1 |
| | protein tau | |
| Phoca vitulina | Microtubule associated | XM_032429419.1 |
| | protein tau | |
| Phoca vitulina | Microtubule associated | XM_032429418.1 |
| | protein tau | |
| Ursus arctos | Microtubule associated | XM_044389174.3 |
| | protein tau | |
| Hyaena hyaena | Microtubule associated | XM_039250673.1 |
| | protein tau | |
| Onychomys torridus | Microtubule associated | XM_036195370.1 |
| | protein tau | |
| Peromyscus maniculatus | Microtubule associated | XM_006970544.3 |
| bairdii | protein tau | |
| Neogale vison | Microtubule associated | XM_044247704.1 |
| | protein tau | |
| Vulpes lagopus | Microtubule associated | XM_041724694.1 |
| | protein tau | |
| Mustela lutreola | Microtubule associated | XM_059148051.1 |
| | protein tau | |
| Dipodomys ordii | Microtubule associated | XM_013033320.1 |
| | protein tau | |
| Ceratotherium simum | Microtubule associated | XM_004432605.2 |
| simum | protein tau | |
| Leptonychotes weddellii | Microtubule associated | XM_006734332.2 |
| | protein tau | |
| Talpa occidentalis | Microtubule associated | XM_037493981.2 |
| | protein tau | |
| Arvicanthis niloticus | Microtubule associated | XM_034505167.1 |
| | protein tau | |
| Canis lupus dingo | Microtubule associated | XM_025436809.3 |
| | protein tau | |
| Canis lupus familiaris | Microtubule associated | XM_038675008.1 |
| | protein tau | |
| Mustela erminea | Microtubule associated | XM_032321032.1 |
| | protein tau | |
| Mustela nigripes | Microtubule associated | XM_059379297.1 |
| | protein tau | |
| Mustela putorius furo | Microtubule associated | XM_013045912.2 |
| | protein tau | |
| Marmota flaviventris | Microtubule associated | XM_027921782.1 |
| | protein tau | |
| Myodes glareolus | Microtubule associated | XM_048445903.1 |
| | protein tau | |
| Rattus norvegicus | Microtubule associated | MZ604977.1 |
| | protein tau | |
| Rattus norvegicus | Microtubule associated | XM_008768277.3 |
| | protein tau | |
| Meles meles | Microtubule associated | XM_045984606.1 |
| | protein tau | |
| Fukomys damarensis | Microtubule associated | XM_010637469.1 |
| | protein tau | |
| Marmota monax | Microtubule associated | XM_046421158.2 |
| | protein tau | |
| Microtus ochrogaster | Microtubule associated | XM_005369396.2 |
| | protein tau | |
| Panthera onca | Microtubule associated | XM_060645817.1 |
| | protein tau | |
| Panthera pardus | Microtubule associated | XM_019437501.2 |
| | protein tau | |
| Microtus oregoni | Microtubule associated | XM_041635538.1 |
| | protein tau | |
| Nyctereutes procyonoides | Microtubule associated | XM_055321793.1 |
| | protein tau | |
| Elephas maximus | Microtubule associated | XM_049861634.1 |
| indicus | protein tau | |
| Equus quagga | Microtubule associated | XM_046675026.1 |
| | protein tau | |
| Sus scrofa | Microtubule associated | KC473498.1 |
| | protein tau | |
| Lontra canadensis | Microtubule associated | XM_032870942.1 |
| | protein tau | |
| Mesocricetus auratus | Microtubule associated | XM_040749879.1 |
| | protein tau | |
| Marmota marmota | Microtubule associated | XM_015507200.2 |
| marmota | protein tau | |
| Lynx canadensis | Microtubule associated | XM_030296037.1 |
| | protein tau | |
| Neofelis nebulosa | Microtubule associated | XM_058705970.1 |
| | protein tau | |
| Acinonyx jubatus | Microtubule associated | XM_027052029.2 |
| | protein tau | |
| Panthera uncia | Microtubule associated | XM_049637605.1 |
| | protein tau | |
| Leopardus geoffroyi | Microtubule associated | XM_045489310.1 |
| | protein tau | |

In one aspect, the DNA constructs disclosed herein incorporate a gene that encodes adipose triglyceride lipase, which is also known as patatin-like phospholipase domain-containing protein 2. In a further aspect, adipose triglyceride lipase is an enzyme that catalyzes the first reaction of lipolysis by breaking down triacylglycerols to diacylglycerols. In an aspect, defects in adipose triglyceride lipase lead to lipid storage diseases due to the accumulation of triacylglycerols. Adipose triglyceride lipase is regulated by insulin and regulation of this enzyme is believed to be involved in various metabolic disorders.

In one aspect, the gene that encodes adipose triglyceride lipase is isolated from a mammal such as, for example, a human, chimpanzee, Western lowland gorilla, bonobo, silvery gibbon, siamang, Southern pig-tailed macaque, Sumatran orangutan, Northern white-cheeked gibbon, Indochinese rhesus macaque, gelada, crab-eating macaque, Francois' langur, golden snub-nosed monkey, Tibetan macaque, black-and-white snub-nosed monkey, green monkey, Bornean orangutan, sooty mangabey, Ugandan red colobus, olive baboon, African woodland thicket rat, Nancy Ma's night monkey, African grass rat, Southern multimammate mouse, golden spiny mouse, Mongolian gerbil, Gairdner's shrewmouse, fat sand rat, Middle East blind mole rat, Panamanian white-faced capuchin, Eastern gray squirrel, cactus mouse, Chinese hamster, wood mouse, black-capped squirrel monkey, Ryukyu mouse, tufted capuchin, cape golden mole, common marmoset, California deermouse, Angola colobus, American beaver, Southern grasshopper mouse, bank vole, Sunda slow loris, Roborovski dwarf hamster, Syrian hamster, great roundleaf bat, white-footed mouse, yellow-bellied marmot, groundhog, Eastern deer mouse, Eurasian otter, Arctic ground squirrel, Blainville's beaked whale, Chinese rufous horseshoe bat, plains zebra, common vampire bat, thirteen-lined ground squirrel, Northern greater galago, African wild ass, European mink, lesser Egyptian jerboa, American mink, Parnell's mustached bat, ring-tailed lemur, greater horseshoe bat, horse, stoat, elk, velvety free-tailed bat, North American river otter, sea otter, striped hyena, or tiger In a further aspect, the gene that encodes adipose triglyceride lipase has SEQ ID NO. 3 or at least 70% homology thereto, at least 75% homology thereto, at least 80% homology thereto, at least 85% homology thereto, at least 90% homology thereto, at least 95% homology thereto, or at least 99% homology thereto.

Other sequences encoding adipose triglyceride lipase or related or homologous genes can be identified in a database such as, for example, GenBank. In one aspect, the gene that encodes adipose triglyceride lipase is isolated from Homo sapiens and can be identified by the GI number AY894804.1 in the GenBank database. In another aspect, sequences useful herein include those with GI numbers listed in Table 3:

TABLE 3
Adipose Triglyceride Lipase

| Source Organism | Sequence Description | GI Number |
|---|---|---|
| Homo sapiens | Adipose triglyceride lipase | AY894804.1 |
| Homo sapiens | Patatin-like phospholipase domain | NM_020376.4 |
| Human ORFeome Gateway | Synthetic construct | LT741424.1 |
| entry vector | | |
| synthetic construct | Patatin-like phospholipase domain | KJ899303.1 |
| Homo sapiens | Patatin-like phospholipase domain | KF601369.1 |
| synthetic construct | Patatin-like phospholipase domain | AB527798.1 |
| Homo sapiens | Patatin-like phospholipase domain | BC017280.2 |
| Homo sapiens | Patatin-like phospholipase domain | KF833265.1 |
| Homo sapiens | Patatin-like phospholipase domain | KF833264.1 |
| Homo sapiens | Patatin-like phospholipase domain | JF279442.1 |
| Homo sapiens | Patatin-like phospholipase domain | JF279441.1 |
| Homo sapiens | Patatin-like phospholipase domain | HQ651812.1 |
| Pan troglodytes | Patatin-like phospholipase domain | XM_016920065.3 |
| Homo sapiens | Transport-secretion protein 2.2 | AJ278476.1 |
| Homo sapiens | Transport-secretion protein 2.1 | AJ278475.1 |
| Gorilla gorilla gorilla | Patatin-like phospholipase domain | XM_055355784.1 |
| Gorilla gorilla gorilla | Patatin-like phospholipase domain | XM_004050383.4 |
| Pan paniscus | Patatin-like phospholipase domain | XM_034931870.2 |
| Homo sapiens | Unknown liver protein | X56789.1 |
| Hylobates moloch | Patatin-like phospholipase domain | XM_032149346.2 |
| Symphalangus syndactylus | Patatin-like phospholipase domain | XM_055260076.1 |
| Macaca nemestrina | Patatin-like phospholipase domain | XM_011762350.2 |
| Pongo abelii | Patatin-like phospholipase domain | XM_024255426.2 |
| Nomascus leucogenys | Patatin-like phospholipase domain | XM_030801978.1 |
| Macaca mulatta | Patatin-like phospholipase domain | XM_015113281.2 |
| Theropithecus gelada | Patatin-like phospholipase domain | XM_025358186.1 |
| Macaca fascicularis | Patatin-like phospholipase domain | XM_015434237.2 |
| Trachypithecus francoisi | Patatin-like phospholipase domain | XM_033204071.1 |
| Rhinopithecus roxellana | Patatin-like phospholipase domain | XM_010388286.2 |
| Rhinopithecus roxellana | Patatin-like phospholipase domain | XM_010388211.2 |
| Macaca thibetana thibetana | Patatin-like phospholipase domain | XM_050757872.1 |
| Rhinopithecus bieti | Patatin-like phospholipase domain | XM_017848603.1 |
| Rhinopithecus bieti | Patatin-like phospholipase domain | XM_017848602.1 |
| Chlorocebus sabaeus | Patatin-like phospholipase domain | XM_007995471.2 |
| Pongo pygmaeus | Patatin-like phospholipase domain | XM_054439008.1 |
| Cercocebus atys | Patatin-like phospholipase domain | XM_012041049.1 |
| Piliocolobus tephrosceles | Patatin-like phospholipase domain | XM_023183379.1 |
| Papio anubis | Patatin-like phospholipase domain | XM_009185251.4 |
| Grammomys surdaster | Patatin-like phospholipase domain | XM_028786881.1 |
| Aotus nancymaae | Patatin-like phospholipase domain | XM_021677035.1 |
| Arvicanthis niloticus | Patatin-like phospholipase domain | XM_034503146.1 |
| Homo sapiens | Unknown protein | AF055000.1 |
| Mastomys coucha | Patatin-like phospholipase domain | XM_031388860.1 |
| Acomys russatus | Patatin-like phospholipase domain | XM_051145654.1 |
| Meriones unguiculatus | Patatin-like phospholipase domain | XM_021646229.2 |
| Mus pahari | Patatin-like phospholipase domain | XM_021198928.2 |
| Psammomys obesus | Patatin-like phospholipase domain | XM_055606888.1 |
| Nannospalax galili | Patatin-like phospholipase domain | XM_008854416.3 |
| Cebus imitator | Patatin-like phospholipase domain | XM_017507810.2 |
| Sciurus carolinensis | Patatin-like phospholipase domain | XM_047519471.1 |
| Peromyscus eremicus | Patatin-like phospholipase domain | XM_059264516.1 |
| Cricetulus griseus | Patatin-like phospholipase domain | XM_027408922.2 |
| Cricetulus griseus | Patatin-like phospholipase domain | XM_003509767.5 |
| Apodemus sylvaticus | Patatin-like phospholipase domain | XM_052195329.1 |
| Saimiri boliviensis boliviensis | Patatin-like phospholipase domain | XM_039471238.1 |
| Mus caroli | Patatin-like phospholipase domain | XM_021167897.2 |
| Sapajus apella | Patatin-like phospholipase domain | XM_032270536.1 |
| Chrysochloris asiatica | Patatin-like phospholipase domain | XM_006877000.1 |
| Callithrix jacchus | Patatin-like phospholipase domain | XM_009008751.4 |
| Peromyscus californicus insignis | Patatin-like phospholipase domain | XM_052714694.1 |
| Colobus angolensis palliatus | Patatin-like phospholipase domain | XM_011930142.1 |
| Castor canadensis | Patatin-like phospholipase domain | XM_020182472.1 |
| Onychomys torridus | Patatin-like phospholipase domain | XM_036176930.1 |
| Myodes glareolus | Patatin-like phospholipase domain | XM_048457986.1 |
| Nycticebus coucang | Patatin-like phospholipase domain | XM_053562712.1 |
| Phodopus roborovskii | Patatin-like phospholipase domain | XM_051193373.1 |
| Mesocricetus auratus | Patatin-like phospholipase domain | XM_005064203.4 |
| Hipposideros armiger | Patatin-like phospholipase domain | XM_019631122.1 |
| Peromyscus leucopus | Patatin-like phospholipase domain | XM_028870327.2 |
| Marmota flaviventris | Patatin-like phospholipase domain | XM_027953504.1 |
| Marmota monax | Patatin-like phospholipase domain | XM_046444737.2 |
| Peromyscus maniculatus bairdii | Patatin-like phospholipase domain | XM_006977231.3 |
| Lutra lutra | Patatin-like phospholipase domain | XM_047690532.1 |
| Urocitellus parryii | Patatin-like phospholipase domain | XM_026379346.1 |
| Urocitellus parryii | Patatin-like phospholipase domain | XM_026379345.1 |
| Mesoplodon densirostris | Patatin-like phospholipase domain | XM_060104505.1 |
| Rhinolophus sinicus | Patatin-like phospholipase domain | XM_019714780.1 |
| Rhinolophus sinicus | Patatin-like phospholipase domain | XM_019714779.1 |
| Equus quagga | Patatin-like phospholipase domain | XM_046644745.1 |
| Desmodus rotundus | Patatin-like phospholipase domain | XM_053924746.1 |
| Ictidomys tridecemlineatus | Patatin-like phospholipase domain | XM_005341631.2 |
| Otolemur garnettii | Patatin-like phospholipase domain | XM_012813320.2 |
| Equus asinus | Patatin-like phospholipase domain | XM_044750133.1 |
| Equus asinus | Patatin-like phospholipase domain | XM_044750132.1 |
| Mustela lutreola | Patatin-like phospholipase domain | XM_059151933.1 |
| Jaculus jaculus | Patatin-like phospholipase domain | XM_004654180.2 |
| Neogale vison | Patatin-like phospholipase domain | XM_044259920.1 |
| Pteronotus parnellii | Patatin-like phospholipase domain | XM_054576810.1 |
| mesoamericanus | | |
| Lemur catta | Patatin-like phospholipase domain | XM_045558535.1 |
| Rhinolophus ferrumequinum | Patatin-like phospholipase domain | XM_033119322.1 |
| Equus caballus | Patatin-like phospholipase domain | XM_023654788.1 |
| Mustela erminea | Patatin-like phospholipase domain | XM_032358385.1 |
| Cervus canadensis | Patatin-like phospholipase domain | XM_043451436.1 |
| Cervus canadensis | Patatin-like phospholipase domain | XM_043451434.1 |
| Molossus molossus | Patatin-like phospholipase domain | XM_036281160.1 |
| Lontra canadensis | Patatin-like phospholipase domain | XM_032879824.1 |
| Enhydra lutris kenyoni | Patatin-like phospholipase domain | XM_022507457.1 |
| Hyaena hyaena | Patatin-like phospholipase domain | XM_039221551.1 |
| Panthera tigris | Patatin-like phospholipase domain | XM_042958627.1 |
| Homo sapiens | Patatin-like phospholipase domain | BC011958.1 |

In one aspect, the DNA constructs disclosed herein incorporate a gene that encodes acyl-CoA dehydrogenase. In a further aspect, acyl-CoA dehydrogenase is an enzyme that catalyzes B-oxidation of fatty acids in the mitochondria. Acyl-CoA dehydrogenase requires an active site glutamate residue and a flavin adenine dinucleotide co-factor in order to function, and introduces a trans double bond between C2 and C3 of an acyl-CoA thioester substrate. In one aspect, medium chain acyl-CoA dehydrogenase is commonly deficient in metabolic disorders in animals.

In one aspect, the gene that encodes acyl-CoA dehydrogenase is isolated from a mammal such as, for example, a bonobo, human, Western lowland gorilla, chimpanzee, Sumatran orangutan, Bornean orangutan, Northern white-cheeked gibbon, silvery gibbon, Tibetan macaque, Indochinese rhesus macaque, green monkey, crab-eating macaque, olive baboon, Southern pig-tailed macaque, sooty mangabey, tufted capuchin, Panamanian white-faced capuchin, Gelada, common marmoset, Ugandan red colobus, Angola colobus, golden snub-nosed monkey, black-and-white snub-nosed monkey, Francois' langur, black-capped squirrel monkey, Arctic ground squirrel, Alpine marmot, common shrew, yellow-bellied marmot, groundhog, gray mouse lemur, Chinese tree shrew, Nancy Ma's night monkey, giant panda, or American pika. In a further aspect, the gene that encodes acyl-CoA dehydrogenase has SEQ ID NO. 4 or at least 70% homology thereto, at least 75% homology thereto, at least 80% homology thereto, at least 85% homology thereto, at least 90% homology thereto, at least 95% homology thereto, or at least 99% homology thereto.

Other sequences encoding acyl-CoA dehydrogenase or related or homologous genes can be identified in a database such as, for example, GenBank. In one aspect, the gene that encodes acyl-CoA dehydrogenase is isolated from Pan paniscus and can be identified by the GI number XM 003830572.4 in the GenBank database. In another aspect, sequences useful herein include those with GI numbers listed in Table 4:

TABLE 4
Acyl-CoA Dehydrogenase

| Source Organism | Sequence Description | GI Number |
|---|---|---|
| Pan paniscus | Acyl-CoA dehydrogenase | XM_003830572.4 |
| | medium chain | |
| Homo sapiens | Acyl-CoA dehydrogenase | NM_000016.6 |
| | medium chain | |
| Homo sapiens | Testicular tissue | HM005320.1 |
| | protein Li 7 | |
| Homo sapiens | Acyl-CoA dehydrogenase | AK312629.1 |
| | medium chain | |
| Homo sapiens | Acyl-CoA dehydrogenase | BC005377.1 |
| | medium chain | |
| Homo sapiens | Acyl-CoA dehydrogenase | M16827.1 |
| | medium chain | |
| Human ORFeome Gateway | Synthetic construct | LT740286.1 |
| entry vector | | |
| synthetic construct | Acyl-CoA dehydrogenase | KR710153.1 |
| | medium chain | |
| synthetic construct | Acyl-CoA dehydrogenase | KJ890612.1 |
| | medium chain | |
| synthetic construct | Acyl-CoA dehydrogenase | DQ895710.2 |
| | medium chain | |
| synthetic construct | Acyl-CoA dehydrogenase | AY892507.1 |
| | medium chain | |
| Gorilla gorilla gorilla | Acyl-CoA dehydrogenase | XM_019019925.2 |
| | medium chain | |
| Homo sapiens | Acyl-CoA dehydrogenase | KU177876.1 |
| | medium chain | |
| synthetic construct | Acyl-CoA dehydrogenase | KR710155.1 |
| | medium chain | |
| synthetic construct | Acyl-CoA dehydrogenase | KR710154.1 |
| | medium chain | |
| synthetic construct | Acyl-CoA dehydrogenase | KR710152.1 |
| | medium chain | |
| synthetic construct | Acyl-CoA dehydrogenase | DQ892341.2 |
| | medium chain | |
| Pan troglodytes | Acyl-CoA dehydrogenase | XM_054664818.1 |
| | medium chain | |
| Pan troglodytes | Acyl-CoA dehydrogenase | NM_001110816.1 |
| | medium chain | |
| Homo sapiens | Acyl-CoA dehydrogenase | NM_001127328.3 |
| | medium chain | |
| Pan troglodytes | Acyl-CoA dehydrogenase | XM_054664897.1 |
| | medium chain | |
| Homo sapiens | Acyl-CoA dehydrogenase | AF251043.1 |
| | medium chain | |
| Pongo abelii | Acyl-CoA dehydrogenase | XM_009248888.3 |
| | medium chain | |
| Pongo pygmaeus | Acyl-CoA dehydrogenase | XM_054494536.1 |
| | medium chain | |
| Pongo abelii | Acyl-CoA dehydrogenase | XM_024245437.2 |
| | medium chain | |
| Pongo pygmaeus | Acyl-CoA dehydrogenase | XM_054494526.1 |
| | medium chain | |
| Nomascus leucogenys | Acyl-CoA dehydrogenase | XM_003260214.3 |
| | medium chain | |
| Pongo abelii | Acyl-CoA dehydrogenase | XM_054532991.1 |
| | medium chain | |
| Hylobates moloch | Acyl-CoA dehydrogenase | XM_032155563.2 |
| | medium chain | |
| Pan paniscus | Acyl-CoA dehydrogenase | XM_034967796.2 |
| | medium chain | |
| Homo sapiens | Similar to acyl-CoA | AK301717.1 |
| | dehydrogenase medium chain | |
| Homo sapiens | Acyl-CoA dehydrogenase | NM_001286042.2 |
| | medium chain | |
| Homo sapiens | Similar to acyl-CoA | AK315946.1 |
| | dehydrogenase medium chain | |
| Macaca thibetana | Acyl-CoA dehydrogenase | XM_050805117.1 |
| thibetana | medium chain | |
| Gorilla gorilla | Acyl-CoA dehydrogenase | XM_055367404.1 |
| gorilla | medium chain | |
| Pan troglodytes | Acyl-CoA dehydrogenase | XM_024353409.2 |
| | medium chain | |
| Macaca mulatta | Acyl-CoA dehydrogenase | XM_001101274.4 |
| | medium chain | |
| Homo sapiens | Acyl-CoA dehydrogenase | KU177877.1 |
| | medium chain | |
| Pongo abelii | Acyl-CoA dehydrogenase | XM_054532990.1 |
| | medium chain | |
| Chlorocebus sabaeus | Acyl-CoA dehydrogenase | XM_007978258.2 |
| | medium chain | |
| Macaca fascicularis | Acyl-CoA dehydrogenase | AB171682.1 |
| | medium chain | |
| Macaca fascicularis | Acyl-CoA dehydrogenase | AB169627.1 |
| | medium chain | |
| Papio anubis | Acyl-CoA dehydrogenase | XM_021943129.2 |
| | medium chain | |
| Macaca nemestrina | Acyl-CoA dehydrogenase | XM_011742871.2 |
| | medium chain | |
| Cercocebus atys | Acyl-CoA dehydrogenase | XM_012082207.1 |
| | medium chain | |
| Macaca fascicularis | Acyl-CoA dehydrogenase | XM_005542992.3 |
| | medium chain | |
| Macaca fascicularis | Acyl-CoA dehydrogenase | AB083301.2 |
| | medium chain | |
| Sapajus apella | Acyl-CoA dehydrogenase | XM_032296571.1 |
| | medium chain | |
| Cebus imitator | Acyl-CoA dehydrogenase | XM_017531538.2 |
| | medium chain | |
| Cebus imitator | Acyl-CoA dehydrogenase | XM_017531530.2 |
| | medium chain | |
| Pongo abelii | Acyl-CoA dehydrogenase | XM_024245449.2 |
| | medium chain | |
| Theropithecus gelada | Acyl-CoA dehydrogenase | XM_025385092.1 |
| | medium chain | |
| Chlorocebus sabaeus | Acyl-CoA dehydrogenase | XM_038001860.1 |
| | medium chain | |
| Pongo pygmaeus | Acyl-CoA dehydrogenase | XM_054494556.1 |
| | medium chain | |
| Callithrix jacchus | Acyl-CoA dehydrogenase | XM_035251910.2 |
| | medium chain | |
| Macaca fascicularis | Similar to acyl-CoA | AB170574.1 |
| | dehydrogenase medium chain | |
| Cercocebus atys | Acyl-CoA dehydrogenase | XM_012082206.1 |
| | medium chain | |
| Piliocolobus tephrosceles | Acyl-CoA dehydrogenase | XM_023209325.2 |
| | medium chain | |
| Colobus angolensis | Acyl-CoA dehydrogenase | XM_011936938.1 |
| palliatus | medium chain | |
| Macaca fascicularis | Acyl-CoA dehydrogenase | XM_015433969.2 |
| | medium chain | |
| Theropithecus gelada | Acyl-CoA dehydrogenase | XM_025385110.1 |
| | medium chain | |
| Homo sapiens | Acyl-CoA dehydrogenase | AK301039.1 |
| | medium chain | |
| Rhinopithecus roxellana | Acyl-CoA dehydrogenase | XM_010368597.2 |
| | medium chain | |
| Rhinopithecus bieti | Acyl-CoA dehydrogenase | XM_017851594.1 |
| | medium chain | |
| Pongo abelii | Acyl-CoA dehydrogenase | XM_024245444.2 |
| | medium chain | |
| Pongo pygmaeus | Acyl-CoA dehydrogenase | XM_054494545.1 |
| | medium chain | |
| Hylobates moloch | Acyl-CoA dehydrogenase | XM_032155564.2 |
| | medium chain | |
| Nomascus leucogenys | Acyl-CoA dehydrogenase | XM_012500493.1 |
| | medium chain | |
| Piliocolobus tephrosceles | Acyl-CoA dehydrogenase | XM_023209309.2 |
| | medium chain | |
| Theropithecus gelada | Acyl-CoA dehydrogenase | XM_025385082.1 |
| | medium chain | |
| Piliocolobus tephrosceles | Acyl-CoA dehydrogenase | XM_023209319.1 |
| | medium chain | |
| Callithrix jacchus | Acyl-CoA dehydrogenase | XM_035251912.2 |
| | medium chain | |
| Cebus imitator | Acyl-CoA dehydrogenase | XM_037739628.1 |
| | medium chain | |
| Colobus angolensis | Acyl-CoA dehydrogenase | XM_011936937.1 |
| palliatus | medium chain | |
| Trachypithecus | Acyl-CoA dehydrogenase | XM_033225326.1 |
| francoisi | medium chain | |
| Sapajus apella | Acyl-CoA dehydrogenase | XM_032296573.1 |
| | medium chain | |
| Sapajus apella | Acyl-CoA dehydrogenase | XM_032296572.1 |
| | medium chain | |
| Cebus imitator | Acyl-CoA dehydrogenase | XM_017531556.2 |
| | medium chain | |
| Cebus imitator | Acyl-CoA dehydrogenase | XM_017531546.2 |
| | medium chain | |
| Callithrix jacchus | Acyl-CoA dehydrogenase | XM_035251916.2 |
| | medium chain | |
| Saimiri boliviensis | Acyl-CoA dehydrogenase | XM_039473607.1 |
| boliviensis | medium chain | |
| Callithrix jacchus | Acyl-CoA dehydrogenase | XM_035251915.2 |
| | medium chain | |
| Callithrix jacchus | Acyl-CoA dehydrogenase | XM_035251911.2 |
| | medium chain | |
| Cercocebus atys | Acyl-CoA dehydrogenase | XM_012082208.1 |
| | medium chain | |
| Callithrix jacchus | Acyl-CoA dehydrogenase | XM_035251917.2 |
| | medium chain | |
| Theropithecus gelada | Acyl-CoA dehydrogenase | XM_025385101.1 |
| | medium chain | |
| Piliocolobus tephrosceles | Acyl-CoA dehydrogenase | XM_023209331.1 |
| | medium chain | |
| Rhinopithecus roxellana | Acyl-CoA dehydrogenase | XM_030942197.1 |
| | medium chain | |
| Colobus angolensis | Acyl-CoA dehydrogenase | XM_011936939.1 |
| palliatus | medium chain | |
| Urocitellus parryii | Acyl-CoA dehydrogenase | XM_026409908.1 |
| | medium chain | |
| Trachypithecus | Acyl-CoA dehydrogenase | XM_033225328.1 |
| francoisi | medium chain | |
| Marmota marmota | Acyl-CoA dehydrogenase | XM_015490525.2 |
| marmota | medium chain | |
| Sorex araneus | Acyl-CoA dehydrogenase | XM_004603339.2 |
| | medium chain | |
| Marmota flaviventris | Acyl-CoA dehydrogenase | XM_027935156.1 |
| | medium chain | |
| Marmota monax | Acyl-CoA dehydrogenase | XM_046443162.2 |
| | medium chain | |
| Microcebus murinus | Acyl-CoA dehydrogenase | XM_012787789.2 |
| | medium chain | |
| Tupaia chinensis | Acyl-CoA dehydrogenase | XM_006146055.2 |
| | medium chain | |
| Aotus nancymaae | Acyl-CoA dehydrogenase | XM_021667744.1 |
| | medium chain | |
| Ailuropoda melanoleuca | Acyl-CoA dehydrogenase | XM_019797170.2 |
| | medium chain | |
| Ochotona princeps | Acyl-CoA dehydrogenase | XM_058658175.1 |
| | medium chain | |

In one aspect, the DNA constructs disclosed herein incorporate a gene that encodes O-GlcNAcase ((protein)-3-O-(N-acetyl-D-glucosaminyl)-L-serine/threonine N-acetylglucosaminyl hydrolase). In a further aspect, O-GlcNAcase is an enzyme that catalyzes the removal of N-acetyl-D-glucosamine (GlcNAc) groups from serine and threonine residues that have been post-translationally modified to include the same. When tau protein is hyperphosphorylated, tau tangles form, leading to neurodegenerative diseases. In one aspect, regulating O-GlcNAcase may provide a target for inhibiting the formation of tau tangles.

In one aspect, the gene that encodes O-GlcNAcase is isolated from a mammal such as, for example, a brown rat, Eastern gray squirrel, Pacific pocket mouse, groundhog, Alpine marmot, wild boar, yellow-bellied marmot, Arctic ground squirrel, thirteen-lined ground squirrel, common warthog, jaguar, lion, horse, Przewalski's horse, lesser Egyptian jerboa, house mouse, European hedgehog, black rat, leopard, snow leopard, Geoffroy's cat, jaguarundi, Pygmy sperm whale, South-central black rhinoceros, Southern white rhinoceros, sea otter, Ord's kangaroo rat, Eurasian otter, Canada lynx, American beaver, bobcat, plains zebra, leopard cat, tiger, Ryukyu mouse, African grass rat, Chinese tree shrew, clouded leopard, cheetah, fishing cat, African wild ass, Jamaican fruit bat, European mink, European badger, ferret, cat, Sunda slow loris, Gairdner's shrewmouse, black-footed ferret, banner-tailed kangaroo rat, African woodland thicket rat, naked mole-rat, stoat, American mink, velvety free-tailed bat, Egyptian fruit bat, North American river otter, dwarf musk deer, Mongolian gerbil, greater spear-nosed bat, golden spiny mouse, beluga whale, olive baboon, Coquerel's sifaka, ring-tailed lemur, pale spear-nosed bat, smoky shrew, Parnell's mustached bat, Sturnira hondurensis, common bottlenose dolphin, narwhal, long-finned pilot whale, short-beaked common dolphin, blue whale, or striped hyena. In a further aspect, the gene that encodes O-GlcNAcase has SEQ ID NO. 5 or at least 70% homology thereto, at least 75% homology thereto, at least 80% homology thereto, at least 85% homology thereto, at least 90% homology thereto, at least 95% homology thereto, or at least 99% homology thereto.

Other sequences encoding O-GlcNAcase or related or homologous genes can be identified in a database such as, for example, GenBank. In one aspect, the gene that encodes O-GlcNAcase is isolated from Rattus norvegicus and can be identified by the GI number AY039679.1 in the GenBank database. In another aspect, sequences useful herein include those with GI numbers listed in Table 5:

TABLE 5
O-GlcNAcase

| Source Organism | Sequence Description | GI Number |
|---|---|---|
| Rattus norvegicus | O-GlcNAcase | AY039679.1 |
| Rattus norvegicus | O-GlcNAcase | NM_131904.2 |
| Rattus norvegicus | O-GlcNAcase | XM_017588708.2 |
| Sciurus carolinensis | O-GlcNAcase | XM_047552737.1 |
| Perognathus longimembris | O-GlcNAcase | XM_048338399.1 |
| pacificus | | |
| Marmota monax | Kv channel-interacting | XM_046450053.2 |
| | protein 2 | |
| Marmota monax | Kv channel-interacting | XM_058581251.1 |
| | protein 2 | |
| Marmota monax | Kv channel-interacting | XM_046450051.2 |
| | protein 2 | |
| Marmota monax | Kv channel-interacting | XM_058581249.1 |
| | protein 2 | |
| Marmota marmota marmota | O-GlcNAcase | XM_048807209.1 |
| Marmota marmota marmota | O-GlcNAcase | XM_048807208.1 |
| Marmota marmota marmota | O-GlcNAcase | XM_015494690.2 |
| Marmota marmota marmota | O-GlcNAcase | XM_048807207.1 |
| Sus scrofa | Meningioma expressed | XM_003483548.4 |
| | antigen 5 | |
| Marmota flaviventris | O-GlcNAcase | XM_027930170.2 |
| Urocitellus parryii | O-GlcNAcase | XM_026407874.1 |
| Ictidomys tridecemlineatus | O-GlcNAcase | XM_013363728.2 |
| Phacochoerus africanus | O-GlcNAcase | XM_047762791.1 |
| Panthera onca | O-GlcNAcase | XM_060641676.1 |
| Panthera leo | O-GlcNAcase | XM_042907498.1 |
| Panthera leo | O-GlcNAcase | XM_042907497.1 |
| Equus caballus | Meningioma expressed | XM_023640514.1 |
| | antigen 5 | |
| Equus caballus | Meningioma expressed | XM_001499535.5 |
| | antigen 5 | |
| Equus przewalskii | Meningioma expressed | XM_008520873.1 |
| | antigen 5 | |
| Jaculus jaculus | O-GlcNAcase | XM_045146582.1 |
| Jaculus jaculus | O-GlcNAcase | XM_045146575.1 |
| Mus musculus | Cytosolic β-N- | AF132214.1 |
| | acetylglucosaminidase | |
| Erinaceus europaeus | O-GlcNAcase | XM_007533846.3 |
| Rattus rattus | O-GlcNAcase | XM_032892079.1 |
| Panthera pardus | O-GlcNAcase | XM_019459219.2 |
| Panthera uncia | O-GlcNAcase | XM_049645592.1 |
| Leopardus geoffroyi | O-GlcNAcase | XM_045438657.1 |
| Puma yagouaroundi | O-GlcNAcase | XM_040486236.1 |
| Kogia breviceps | O-GlcNAcase | XM_059054079.1 |
| Diceros bicornis minor | O-GlcNAcase | XM_058543815.1 |
| Ceratotherium | Meningioma expressed | XM_004427981.2 |
| simum simum | antigen 5 | |
| Mus musculus | Meningioma expressed | BC054821.1 |
| | antigen 5 | |
| Enhydra lutris kenyoni | Meningioma expressed | XM_022499195.1 |
| | antigen 5 | |
| Enhydra lutris kenyoni | Meningioma expressed | XM_022499194.1 |
| | antigen 5 | |
| Dipodomys ordii | Meningioma expressed | XM_013017723.1 |
| | antigen 5 | |
| Lutra lutra | O-GlcNAcase | XM_047701770.1 |
| Lutra lutra | O-GlcNAcase | XM_047701769.1 |
| Lynx canadensis | O-GlcNAcase | XM_030335221.1 |
| Castor canadensis | Meningioma expressed | XM_020171238.1 |
| | antigen 5 | |
| Lynx rufus | O-GlcNAcase | XM_047073334.1 |
| Equus quagga | O-GlcNAcase | XM_046652301.1 |
| Prionailurus bengalensis | O-GlcNAcase | XM_043597319.1 |
| Panthera tigris | O-GlcNAcase | XM_042959862.1 |
| Panthera tigris | O-GlcNAcase | XM_042959861.1 |
| Panthera tigris | O-GlcNAcase | XM_042959860.1 |
| Mus caroli | O-GlcNAcase | XM_021151373.2 |
| Mus musculus | O-GlcNAcase | NM_023799.5 |
| Mus musculus | O-GlcNAcase | NM_001403349.1 |
| Arvicanthis niloticus | O-GlcNAcase | XM_034516782.1 |
| Tupaia chinensis | O-GlcNAcase | XM_006166334.3 |
| Neofelis nebulosa | O-GlcNAcase | XM_058697556.1 |
| Acinonyx jubatus | O-GlcNAcase | XM_027062915.2 |
| Prionailurus viverrinus | O-GlcNAcase | XM_047824902.1 |
| Equus asinus | O-GlcNAcase | XM_014837652.2 |
| Artibeus jamaicensis | O-GlcNAcase | XM_037159363.2 |
| Mustela lutreola | O-GlcNAcase | XM_059169127.1 |
| Mustela lutreola | O-GlcNAcase | XM_059169126.1 |
| Meles meles | O-GlcNAcase | XM_046026462.1 |
| Mustela putorius furo | O-GlcNAcase | XM_004749434.3 |
| Mustela putorius furo | O-GlcNAcase | XM_004749433.3 |
| Mus musculus | mKIAA0679 protein | AK129188.1 |
| Felis catus | O-GlcNAcase | XM_003994344.6 |
| Nycticebus coucang | O-GlcNAcase | XM_053583034.1 |
| Mus pahari | O-GlcNAcase | XM_029535279.1 |
| Mustela nigripes | O-GlcNAcase | XM_059398399.1 |
| Dipodomys spectabilis | O-GlcNAcase | XM_042689642.1 |
| Dipodomys spectabilis | O-GlcNAcase | XM_042689641.1 |
| Grammomys surdaster | O-GlcNAcase | XM_028776359.1 |
| Heterocephalus glaber | Meningioma expressed | XM_004866368.3 |
| | antigen 5 | |
| Mustela erminea | O-GlcNAcase | XM_032311920.1 |
| Mustela erminea | O-GlcNAcase | XM_032311919.1 |
| Neogale vison | O-GlcNAcase | XM_044240350.1 |
| Molossus molossus | O-GlcNAcase | XM_036264564.1 |
| Rousettus aegyptiacus | O-GlcNAcase | XM_016120014.2 |
| Lontra canadensis | O-GlcNAcase | XM_032881825.1 |
| Lontra canadensis | O-GlcNAcase | XM_032881820.1 |
| Moschus berezovskii | O-GlcNAcase | XM_055424812.1 |
| Meriones unguiculatus | O-GlcNAcase | XM_021649955.2 |
| Meriones unguiculatus | O-GlcNAcase | XM_060390541.1 |
| Phyllostomus hastatus | O-GlcNAcase | XM_045853766.1 |
| Acomys russatus | O-GlcNAcase | XM_051146672.1 |
| Delphinapterus leucas | O-GlcNAcase | XM_022568972.2 |
| Papio anubis | O-GlcNAcase | XM_003904159.3 |
| Propithecus coquereli | Meningioma expressed | XM_012661710.1 |
| | antigen 5 | |
| Lemur catta | O-GlcNAcase | XM_045568561.1 |
| Phyllostomus discolor | O-GlcNAcase | XM_036027189.1 |
| Sorex fumeus | O-GlcNAcase | XM_056120308.1 |
| Pteronotus parnellii | O-GlcNAcase | XM_054581399.1 |
| mesoamericanus | | |
| Sturnira hondurensis | O-GlcNAcase | XM_037064097.1 |
| Tursiops truncatus | O-GlcNAcase | XM_019939852.2 |
| Monodon monoceros | O-GlcNAcase | XM_029245057.1 |
| Globicephala melas | O-GlcNAcase | XM_030865399.2 |
| Delphinus delphis | O-GlcNAcase | XM_060034213.1 |
| Balaenoptera musculus | O-GlcNAcase | XM_036828180.1 |
| Hyaena hyaena | O-GlcNAcase | XM_039243923.1 |

In one aspect, the DNA construct has the following genetic components: a) a gene that encodes β-amyloid precursor protein, b) a gene that encodes MAPT, c) a gene that encodes adipose triglyceride lipase, d) a gene that encodes an acyl-CoA dehydrogenase, and e) a gene that encodes an O-GlcNAcase.

In another aspect, said construct further includes a) a promoter, b) a terminator or stop sequence, c) a gene that confers resistance to an antibiotic (a “selective marker”), d) a reporter protein, or any combination thereof. Each of these elements is described in further detail below.

In one aspect, the construct includes from 5′ to 3′ the following genetic components in the following order: (1) a gene that encodes β-amyloid precursor protein, (2) a gene that encodes MAPT, (3) a gene that encodes adipose triglyceride lipase, (4) a gene that encodes an acyl-CoA dehydrogenase, and (5) a gene that encodes OGlcNAcase.

In one aspect, the construct includes from 5′ to 3′ the following genetic components in the following order: a gene that encodes β-amyloid precursor protein having SEQ ID NO. 1 or at least 70% homology thereto, a gene that encodes MAPT having SEQ ID NO. 2 or at least 70% homology thereto, a gene that encodes adipose triglyceride lipase having SEQ ID NO. 3 or at least 70% homology thereto, a gene that encodes acyl CoA dehydrogenase having SEQ ID NO. 4 or at least 70% homology thereto, and a gene that encodes OGlcNAcase having SEQ ID NO. 5 or at least 70% homology thereto.

In another aspect, the construct includes from 5′ to 3′ the following genetic components in the following order: (1) a gene that encodes β-amyloid precursor protein; (2) a CYC1 terminator; (3) a GAL1 promoter; (4) a gene that encodes MAPT; (5) a CYC1 terminator; (6) a GAL1 promoter; (7) a gene that encodes adipose triglyceride lipase; (8) a CYC1 terminator; (9) a GAL1 promoter; (10) a gene that encodes acyl CoA dehydrogenase; (11) a CYC1 terminator; (12) a GAL1 promoter; and (12) a gene that encodes OGlcNAcase.

In another aspect, the construct includes from 5′ to 3′ the following genetic components in the following order: (1) a gene that encodes β-amyloid precursor protein having SEQ ID NO. 1 or at least 90% homology thereto; (2) a CYC1 terminator; (3) a GAL1 promoter; (4) a gene that encodes MAPT having SEQ ID NO. 2 or at least 90% homology thereto; (5) a CYC1 terminator; (6) a GAL1 promoter; (7) a gene that encodes adipose triglyceride lipase having SEQ ID NO. 3 or at least 90% homology thereto; (8) a CYC1 terminator; (9) a GAL1 promoter; (10) a gene that encodes acyl CoA dehydrogenase having SEQ ID NO. 4 or at least 90% homology thereto; (11) a CYC1 terminator; (12) a GAL1 promoter; and (12) a gene that encodes OGlcNAcase having SEQ ID NO. 5 or at least 90% homology thereto.

In still another aspect, the construct is a pYES2 plasmid having from 5′ to 3′ the following genetic components in the following order: (1) a gene that encodes β-amyloid precursor protein having SEQ ID NO. 1 or at least 70% homology thereto; (2) a CYC1 terminator; (3) a GAL1 promoter; (4) a gene that encodes MAPT having SEQ ID NO. 2 or at least 70% homology thereto; (5) a CYC1 terminator; (6) a GAL1 promoter; (7) a gene that encodes adipose triglyceride lipase having SEQ ID NO. 3 or at least 70% homology thereto; (8) a CYC1 terminator; (9) a GAL1 promoter; (10) a gene that encodes acyl CoA dehydrogenase having SEQ ID NO. 4 or at least 70% homology thereto; (11) a CYC1 terminator; (12) a GAL1 promoter; and (12) a gene that encodes OGlcNAcase having SEQ ID NO. 5 or at least 70% homology thereto. In some aspects, the construct further includes a gene that encodes enhanced green fluorescent protein having SEQ ID NO. 6 or at least 70% homology thereto positioned after the gene that encodes OGlcNAcase.

In another aspect, the DNA construct has SEQ ID NO. 7 or at least 70% homology thereto, at least 75% homology thereto, at least 80% homology thereto, at least 85% homology thereto, at least 90% homology thereto, at least 95% homology thereto, or at least 99% homology thereto.

### Additional Components of the DNA Constructs

In another aspect, said construct further includes a) a promoter, b) a terminator or stop sequence, c) a gene that confers resistance to an antibiotic (a “selective marker”), d) a reporter protein, or any combination thereof.

In one aspect, the construct includes a regulatory sequence. In a further aspect, the regulatory sequence is already incorporated into a vector such as, for example, a plasmid, prior to genetic manipulation of the vector. In another aspect, the regulatory sequence can be incorporated into the vector through the use of restriction enzymes or any other technique known in the art.

In one aspect, the regulatory sequence is a promoter. The term “promoter” refers to a DNA sequence capable of controlling the expression of a coding sequence. In another aspect, the coding sequence to be controlled is located 3′ to the promoter. In still another aspect, the promoter is derived from a native gene. In an alternative aspect, the promoter is composed of multiple elements derived from different genes and/or promoters. A promoter can be assembled from elements found in nature, from artificial and/or synthetic elements, or from a combination thereof. It is understood by those skilled in the art that different promoters can direct the expression of a gene in different tissues or cell types, at different stages of development, in response to different environmental or physiological conditions, and/or in different species. In one aspect, the promoter functions as a switch to activate the expression of a gene.

In one aspect, the promoter is “constitutive.” A constitutive promoter is a promoter that causes a gene to be expressed in most cell types at most times. In another aspect, the promoter is “regulated.” A regulated promoter is a promoter that becomes active in response to a specific stimulus. A promoter may be regulated chemically, such as, for example, in response to the presence or absence of a particular metabolite (e.g., lactose or tryptophan), a metal ion, a molecule secreted by a pathogen, or the like. A promoter also may be regulated physically, such as, for example, in response to heat, cold, water stress, salt stress, oxygen concentration, illumination, wounding, or the like.

Promoters that are useful to drive expression of the nucleotide sequences described herein are numerous and familiar to those skilled in the art. Suitable promoters include, but are not limited to, the following: T3 promoter, T7 promoter, an iron promoter, araBAD promoter, and GAL1 promoter. In a further aspect, the promoter is a native part of the vector used herein. Variants of these promoters are also contemplated. The skilled artisan will be able to use site-directed mutagenesis and/or other mutagenesis techniques to modify the promoters to promote more efficient function. The promoter may be positioned, for example, from 10-100 nucleotides from a ribosomal binding site.

In one aspect, the promoter is a GAL1 promoter. In another aspect, the GAL1 promoter is native to the plasmid used to create the vector. In another aspect, a GAL1 promoter is positioned before the gene that encodes β-amyloid precursor protein, the gene that encodes MAPT, the gene that encodes adipose triglyceride lipase, the gene that encodes acyl-CoA dehydrogenase, the gene that encodes OGlcNAcase, or any combination thereof. In another aspect, the promoter is a GAL1 promoter obtained from or native to the pYES2 plasmid.

In another aspect, the promoter is a T7 promoter. In a further aspect, the T7 promoter is native to the plasmid used to create the vector. In still another aspect, an T7 promoter is positioned before the gene that encodes β-amyloid precursor protein, the gene that encodes MAPT, the gene that encodes adipose triglyceride lipase, the gene that encodes acyl-CoA dehydrogenase, the gene that encodes OGlcNAcase, or any combination thereof.

In one aspect, the regulatory sequence is an operon such as, for example, the LAC operon or LAC operator. As used herein, an “operon” is a segment of DNA containing a group of genes wherein the group is controlled by a single promoter. Genes included in an operon are all transcribed together. In a further aspect, the operon is a LAC operon and can be induced when lactose crosses the cell membrane of the biological device.

In another aspect, the regulatory sequence is a terminator or stop sequence. As used herein, a terminator is a sequence of DNA that marks the end of a gene or operon to be transcribed. In a further aspect, the terminator is an intrinsic terminator or a Rho-dependent transcription terminator. As used herein, an intrinsic terminator is a sequence wherein a hairpin structure can form in the nascent transcript that disrupts the mRNA/DNA/RNA polymerase complex. As used herein, a Rho-dependent transcription terminator requires a Rho factor protein complex to disrupt the mRNA/DNA/RNA polymerase complex. In one aspect, the terminator is an rrnB terminator obtained from or native to the pBAD plasmid. In an alternative aspect, the terminator is a CYC1 terminator obtained from or native to the pYES2 plasmid.

In a further aspect, the regulatory sequence includes both a promoter and a terminator or stop sequence. In a still further aspect, the regulatory sequence can include multiple promoters or terminators. Other regulatory elements, such as enhancers, are also contemplated. Enhancers may be located from about 1 to about 2000 nucleotides in the 5′ direction from the start codon of the DNA to be transcribed, or may be located 3′ to the DNA to be transcribed. Enhancers may be “cis-acting,” that is, located on the same molecule of DNA as the gene whose expression they affect.

### Further Components of the DNA Constructs and Methods for Making Thereof

In another aspect, the vector contains one or more ribosomal binding sites. As used herein, a “ribosomal binding site” or “rbs” is a sequence of nucleotides located 5′ to the start codon of an mRNA that recruits a ribosome to initiate protein translation. In one aspect, the ribosomal binding site can be positioned before one or more or all genes in the DNA construct, or a before a subset of genes in a DNA construct.

In one aspect, when the vector is a plasmid, the plasmid can also contain a multiple cloning site or polylinker. In a further aspect, the polylinker contains recognition sites for multiple restriction enzymes. The polylinker can contain up to 2, 3, 4, 5, 6, 7, 8, 9, 10, 11, 12, 13, 14, 15, 16, 17, 18, 19, 20, or more than 20 recognition sites for restriction enzymes. Further, restriction sites may be added, disabled, or removed as required, using techniques known in the art. In one aspect, the plasmid contains restriction sites for any known restriction enzyme such as, for example, Hindlll, Kpnl, Sacl, BamHI, BstXI, EcoRI, BasBI, Notl, Xhol, Xphl, Xbal, Apal, Sall, Clal, EcoRV, Pstl, Smal, Xmal, Spel, Eagl, Sacll, or any combination thereof. In a further aspect, the plasmid contains more than one recognition site for the same restriction enzyme.

In one aspect, the restriction enzyme can cleave DNA at a palindromic or an asymmetrical restriction site. In a further aspect, the restriction enzyme cleaves DNA to leave blunt ends; in an alternative aspect, the restriction enzyme cleaves DNA to leave “sticky” or overhanging ends. In another aspect, the enzyme can cleave DNA at a distance of from 20 bases to over 1000 bases away from the restriction site. A variety of restriction enzymes are commercially available and their recognition sequences, as well as instructions for use (e.g., amount of DNA needed, precise volumes of reagents, purification techniques, as well as information about salt concentration, pH, optimum temperature, incubation time, and the like) are provided by enzyme manufacturers.

In one aspect, a plasmid with a polylinker containing one or more restriction sites can be digested with one restriction enzyme and a nucleotide sequence of interest can be ligated into the plasmid using a commercially-available DNA ligase enzyme. Several such enzymes are available, often as kits containing all reagents and instructions required for use. In another aspect, a plasmid with a polylinker containing two or more restriction sites can be simultaneously digested with two restriction enzymes and a nucleotide sequence of interest can be ligated into the plasmid using a DNA ligase enzyme. Using two restriction enzymes provides an asymmetric cut in the DNA, allowing for insertion of a nucleotide sequence of interest in a particular direction and/or on a particular strand of the double-stranded plasmid. Since RNA synthesis from a DNA template proceeds from 5′ to 3′, usually starting just after a promoter, the order and direction of elements inserted into a plasmid can be especially important. If a plasmid is to be simultaneously digested with multiple restriction enzymes, these enzymes must be compatible in terms of buffer, salt concentration, and other incubation parameters.

In some aspects, prior to ligation using a ligase enzyme, a plasmid that has been digested with a restriction enzyme is treated with an alkaline phosphatase enzyme to remove 5′ terminal phosphate groups. This prevents self-ligation of the plasmid and thus facilitates ligation of heterologous nucleotide fragments into the plasmid.

In one aspect, different genes can be ligated into a plasmid in one pot. In this aspect, the genes will first be digested with restriction enzymes. In certain aspects, the digestion of genes with restriction enzymes provides multiple pairs of matching 5′ and 3′ overhangs that will spontaneously assemble the genes in the desired order. In another aspect, the genes and components to be incorporated into a plasmid can be assembled into a single insert sequence prior insertion into the plasmid. In a further aspect, a DNA ligase enzyme can be used to assist in the ligation process.

In another aspect, the ligation mix may be incubated in an electromagnetic chamber. In one aspect, the incubation lasts for about 1 minute, about 2 minutes, about 5 minutes, about 10 minutes, about 15 minutes, about 20 minutes, about 30 minutes, or about 1 hour.

The DNA construct described herein can be part of a vector. In general, plasmid vectors containing replicon and control sequences that are derived from species compatible with the host cell are used in connection with the hosts. The vector ordinarily carries a replication site as well as marking sequences that are capable of performing phenotypic selection in transformed cells. Plasmid vectors are well known and commercially available. Such vectors include, but are not limited to, pWLneo, pSV2cat, pOG44, pXT1, PSG, pSVK3, pBSK, pYES, pYES2, pBSKII, pET, pUC, pUC19, pBAD, and pETDuet-1 vectors.

Plasmids are double-stranded, autonomously-replicating, genetic elements that are not integrated into host cell chromosomes. Further, these genetic elements are usually not part of the host cell's central metabolism. In bacteria, plasmids may range from 1 kilobase (kb) to over 200 kb. Plasmids can be engineered to encode a number of useful traits including the production of secondary metabolites, antibiotic resistance, the production of useful proteins, degradation of complex molecules and/or environmental toxins, and others. Plasmids have been the subject of much research in the field of genetic engineering, as plasmids are convenient expression vectors for foreign DNA in, for example, microorganisms. Plasmids generally contain regulatory elements such as promoters and terminators and also usually have independent replication origins. Ideally, plasmids will be present in multiple copier per host cell and will contain selectable markers (such as genes for antibiotic resistance) to show the skilled artisan to select host eels that have been successfully transfected with the plasmids (for example, by growing the host cells in a medium containing the antibiotic).

In one aspect, the vector encodes a selection marker. In a further aspect, the selection marker is a gene that confers resistance to an antibiotic. In certain aspects, during fermentation of host cells transformed with the vector, the cells are contacted with the antibiotic. For example, the antibiotic may be included in the culture medium. Cells that have not been successfully transformed cannot survive in the presence of the antibiotic; only cells containing the vector, which confers antibiotic resistance, can survive. Optimally, only cells containing the vector to be expressed will be cultured, as this will result in the highest production efficiency of the desired gene products (e.g., peptides). Cells that do not contain the vector would otherwise compete with transformed cells for resources. In one aspect, the antibiotic is tetracycline, neomycin, kanamycin, ampicillin, hygromycin, chloramphenicol, amphotericin B, bacitracin, carbapenam, cephalosporin, ethambutol, fluoroquinolones, isonizid, methicillin, oxacillin, vancomycin, streptomycin, quinolines, rifampin, rifampicin, sulfonamides, cephalothin, erythromycin, streptomycin, gentamycin, penicillin, other commonly-used antibiotics, or a combination thereof.

In certain aspects, the DNA construct can include a gene that encodes a reporter protein. The selection of the reporter protein can vary. For example, the reporter protein can be a yellow fluorescent protein, a red fluorescent protein, a green fluorescent protein, or a cyan fluorescent protein. In one aspect, the reporter protein is a green fluorescent protein and the gene that encodes the reporter protein has SEQ ID NO. 6 or at least 70% homology thereto. The amount of fluorescence that is produced can be correlated to the amount of DNA incorporated into the transfected cells. The fluorescence produced can be detected and quantified using techniques known in the art. For example, spectrofluorometers are typically used to measure fluorescence.

The DNA construct described herein can be part of a vector. In one aspect, the vector is a plasmid, a phagemid, a cosmid, a yeast artificial chromosome, a bacterial artificial chromosome, a virus, a phage, or a transposon.

Exemplary methods for producing the DNA constructs described herein are provided in the Examples. Restriction enzymes and purification techniques known in the art can be used to assemble the DNA constructs. Backbone plasmids and synthetic inserts can be mixed together for ligation purposes at different ratios ranging from 1:1, 1:2, 1:3, 1:4, and up to 1:5. In one aspect, the ratio of backbone plasmid to synthetic insert is 1:4. After the vector comprising the DNA construct has been produced, the resulting vector can be incorporated into the host cells using the methods described below.

### Cells and Biological Devices

A variety of different types of cells can be used in the methods described herein. In one aspect, the cells can be wild-type cells (i.e., not genetically-modified). In one aspect, the cells are from an animal such as, for example, a mammal, bird, fish, reptile, amphibian, or invertebrate. In another aspect, the cells are from a plant such as, for example, an agricultural crop, a decorative plant, a woody plant, a medicinal plant, or a combination thereof. In another aspect, the cells are from a multicellular fungus such as, for example, a mushroom, a mycorrhizal fungus, or a commercially-important mold.

In another aspect, the cells include a biological device. A “biological device” is formed when a microbial cell is transfected with a DNA construct. The biological devices are generally composed of microbial host cells, where the host cells are transformed (i.e., genetically-modified) with a DNA construct.

In one aspect, the DNA construct is carried by the expression vector into the cell and is separate from the host cell's genome. In another aspect, the DNA construct is incorporated into the host cell's genome. In still another aspect, incorporation of the DNA construct into the host cell enables the host cell to produce an extract or composition that can remove metals and/or other contaminants from water or petroleum, such as, for example, those disclosed herein. “Heterologous” genes and proteins are genes and proteins that have been experimentally inserted into a cell that are not normally expressed by the cell. A heterologous gene may be cloned or derived from a different cell type or species than the recipient cell or organism. Heterologous genes may be introduced into cells by transduction or transformation.

An “isolated” nucleic acid is one that has been separated from other nucleic acid molecules and/or cellular material (peptides, proteins, lipids, saccharides, and the like) normally present in the natural source of the nucleic acid. An “isolated” nucleic acid may optionally be free of the flanking sequences found on either side of the nucleic acid as it naturally occurs. An isolated nucleic acid can be naturally occurring, can be chemically synthesized, or can be a cDNA molecule (i.e., is synthesized from an mRNA template using reverse transcriptase and DNA polymerase enzymes).

“Transformation” or “transfection” as used herein refers to a process for introducing heterologous DNA into a host cell. Transformation can occur under natural conditions or may be induced using various methods known in the art. Many methods for transformation are known in the art and the skilled practitioner will know how to choose the best transformation method based on the type of cells being transformed. Methods for transformation include, for example, viral infection, electroporation, lipofection, chemical transformation, and particle bombardment. Cells may be stably transformed (i.e., the heterologous DNA is capable of replicating as an autonomous plasmid or as part of the host chromosome) or may be transiently transformed (i.e., the heterologous DNA is expressed only for a limited period of time).

“Competent cells” refers to microbial cells capable of taking up heterologous DNA. Competent cells can be purchased from a commercial source, or cells can be made competent using procedures known in the art. Exemplary procedures for producing competent cells are provided in the Examples.

The host cells as referred to herein include their progeny, which are any and all subsequent generations formed by cell division. It is understood that not all progeny may be identical due to deliberate or inadvertent mutations. A host cell may be “transfected” or “transformed,” which refers to a process by which an exogenous nucleic acid is transferred or introduced into the host cell.

A transformed cell includes the primary subject cell and its progeny. The host cells can be naturally-occurring cells or “recombinant” cells. Recombinant cells are distinguishable from naturally-occurring cells in that naturally-occurring cells do not contain heterologous DNA introduced through molecular cloning procedures. In one aspect, the host cell is a prokaryotic cell such as, for example, Escherichia coli. In other aspects, the host cell is a eukaryotic cell such as, for example, the yeast Saccharomyces cerevisiae. Host cells transformed with the DNA construct described herein are referred to as “biological devices.”

The DNA construct is first delivered into the host cell. In one aspect, the host cells are naturally competent (i.e., able to take up exogenous DNA from the surrounding environment). In another aspect, cells must be treated to induce artificial competence. This delivery may be accomplished in vitro, using well-developed laboratory procedures for transforming cell lines. Transformation of bacterial cell lines can be achieved using a variety of techniques. One method involves calcium chloride. The exposure to the calcium ions renders the cells able to take up the DNA construct. Another method is electroporation. In this technique, a high-voltage electric field is applied briefly to cells, producing transient holes in the membranes of the cells through which the vector containing the DNA construct enters. Another method involves exposing intact yeast cells to alkali cations such as, for example, lithium. In one aspect, this method includes exposing yeast to lithium acetate, polyethylene glycol, and single-stranded DNA such as, for example, salmon sperm DNA. Without wishing to be bound by theory, the single-stranded DNA is thought to bind to the cell wall of the yeast, thereby blocking plasmids from binding. The plasmids are then free to enter the yeast cell. Enzymatic and/or electromagnetic techniques can also be used alone, or in combination with other methods, to transform microbial cells. Exemplary procedures for transforming yeast and bacteria with specific DNA constructs are provided in the Examples. In certain aspects, two or more types of DNA can be incorporated into the host cells. Thus, different metabolites can be produced from the same host cells at enhanced rates.

### Cell Culture

A satisfactory microbiological culture contains available sources of hydrogen donors and acceptors, carbon, nitrogen, sulfur, phosphorus, inorganic salts and, in certain cases, vitamins or other growth-promoting substances. For example, the addition of peptone provides a readily-available source of nitrogen and carbon. Furthermore, the use of different types of media results in different growth rates and different stationary phase densities. A rich media results in a short doubling time and higher cell density at stationary phase. Minimal media results in slow growth and low final cell densities. Efficient agitation and aeration increase final cell densities.

Culturing or fermenting of host cells can be accomplished by any technique known in the art. In one aspect, batch fermentation can be conducted. In batch fermentation, the composition of the culture medium is set at the beginning and the system is closed to future alterations. In some aspects, a limited form of batch fermentation may be carried out, wherein factors such as oxygen concentration and pH are manipulated, but additional carbon is not added. Continuous fermentation methods are also contemplated. In continuous fermentation, equal amounts of a defined medium are continuously added to and removed from a bioreactor. In other aspects, microbial cells are immobilized on a substrate. Fermentation may be carried out on any scale and may include methods in which literal “fermentation” is carried out as well as other culture methods that are non-fermentative.

In one aspect, the microorganisms can be cultured for a period of from 2 days to 2 weeks, or for about 2, about 3, about 4, about 5, about 6, about 7, about 8, about 9, about 10, about 11, about 12, about 13, or about 14 days, where any value can be the lower or upper endpoint of a range (e.g., about 3 days to about 13 days, about 8 days to about 12 days, etc.). In one aspect, the microorganisms are cultured for about 10 days.

In another aspect, the microorganisms can be cultured at any temperature appropriate for the microorganisms, with the understanding that the temperature may vary according to the microorganism (for example, a thermophilic microorganism may require a higher culture temperature than a mesophile). In one aspect, the microorganisms are cultured at a temperature of from about 20 to about 37° C., or are cultured at about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., or about 37° C., where any value can be the lower or upper endpoint of a range, where any value can be the lower or upper endpoint of a range (e.g., about 21° C. to about 36° C., about 25° C. to about 30° C., etc.).

In certain aspects, after culturing the microorganisms for a sufficient time, the microbial cells can be lysed with one or more enzymes. For example, when the microbial cells are fungal, the fungal cells can be lysed with lyticase. In one aspect, the lyticase concentration can be about 500 μL, about 600 μL, about 700 μL, about 800 μL, about 900 μL, or about 1,000 μL per liter of culture, where any value can be the lower or upper endpoint of a range, where any value can be the lower or upper endpoint of a range (e.g., about 500 μL to about 900 μL, about 600 μL to about 800 μL, etc.).

In addition to or in place of enzymes, other components can be used to facilitate lysis of the microbial cells. In one aspect, chitosan can be used in combination with an enzyme to lyse the microbial cells. Chitosan is generally composed of glucosamine units and N-acetylglucosamine units and can be chemically or enzymatically extracted from chitin, which is a component of arthropod exoskeletons and fungal and microbial cell walls. In certain aspects, the chitosan can be acetylated to a specific degree of acetylation. In one aspect, the chitosan is from about 60% to about 100% acetylated, or about 70%, about 75%, about 80%, about 85%, about 90%, about 95%, or about 100% acetylated, where any value can be the lower or upper endpoint of a range, where any value can be the lower or upper endpoint of a range (e.g., about 60% to about 90%, about 70% to about 80%, etc.).

The molecular weight of the chitosan can vary, as well. For example, the chitosan can comprise about 3, 4, 5, 6, 7, 8, 9, 10, 11, 12, 13, 14, 15, 16, 17, 18, 19, or 20 glucosamine units and/or N-acetylglucosamine units, where any value can be the lower or upper endpoint of a range, where any value can be the lower or upper endpoint of a range (e.g., 2 to 19, 3 to 10, 5 to 7, etc.). In one aspect, chitosan can be added until a concentration of about 0.0015%, about 0.0025%, about 0.005%, about 0.0075%, about 0.01%, about 0.015%, about 0.02%, about 0.03%, about 0.04%, or about 0.05%, where any value can be an upper or lower endpoint of a range (e.g., 0.002% to 0.04%, 0.05% to 0.015%, etc.).

In another aspect, cells can first be fermented, for example, in a biofermenter, at a temperature conducive to cell growth. In one aspect, the cells are fermented at 30° C. In a further aspect, the cells are fermented for a time period sufficient to produce the metabolite(s) of interest. In one aspect, the cells are fermented for from 6 hours to 96 hours, or for 6, 12, 18, 24, 30, 36, 42, 48, 54, 60, 66, 72, 78, 84, 90, or about 96 hours, or a combination of any of the foregoing values, or a range encompassing any of the foregoing values. In one aspect, during fermentation, a micro-current can be applied to the cells as described above. In some aspects, the micro-current is applied for the entire culture period. In another aspect, the micro-current is applied for only a part of the culture period, or for several non-consecutive parts of the culture period. In one aspect, the micro-current is the same throughout the entire culture period. In an alternative aspect, the micro-current is varied during the culture period.

Exemplary methods for culturing cells and/or the biological devices disclosed herein are provided in the Examples.

### Culture Medium

In some aspects, the cells are suspended in a culture medium. In another aspect, the culture medium can be Dulbecco's Modified Eagle Medium (DMEM), RPMI 1640, Minimal Essential Medium (MEM), Eagle's Minimal Essential Medium (EMEM), Iscove's Modified Dulbecco's Medium (IMDM), DMEM/F12 Medium, Murashige and Skoog (MS) medium, White's medium, Agrobacterium minimal medium, Banana AGS basal medium, Blaydes basal medium, Bold's basal medium, Chu (N6) medium, De Greef and Jacobs Medium, DKW basal medium, Economou and Read basal medium, Gamborg (B5) medium, Gresshoff and Doy medium, Heller medium, Hoagland complete medium, Jensen's medium, Kao and Michayluk medium, Litvay medium, NB basal medium, Nitsch medium. NLN medium, Quoirin and Lepoivre medium, Schenk and Hildebrandt medium, TAP medium, TM4G medium, Vacin and Went medium, wheat callus induction medium, Luria Bertani (LB) broth, terrific broth, tryptic soy broth, minimal salts (M9) medium, SOB medium, SOC medium, yeast malt medium, YPD broth, YNB broth, synthetic complete (SC) medium, YPG medium, Hartwell's complete (HC) medium, or a combination thereof. In one aspect, the culture medium is Luria Bertani (LB) broth or yeast malt medium.

In another aspect, the culture medium can contain supplemental compounds such as, for example, vitamins, nucleosides, nucleotides, amino acids, a carbohydrate, an antibiotic, or a combination thereof.

In one aspect, the culture medium can be a liquid. In another aspect, the methods disclosed herein can be performed in a biofermenter. In an alternative aspect, the cells can be distributed on a substrate. In one aspect, the substrate can be agar, a culture dish, contaminated soil, a wastewater treatment device, mineral ore, a plant organ, a tissue scaffold, or a fermentable material. When the substrate is a plant organ, in some aspects, the plant organ can be a root, leaf, stem, rhizome, tuber, flower, seed, fruit, vegetable, callus, or a combination thereof. When the substrate is a fermentable material, in some aspects, the substrate can be milk, a grain, cabbage, soybeans, fish, or a biomass feedstock. When the substrate is a biomass feedstock, in some aspects, the substrate can be forestry residue, logging residue, sawmill residue, animal manure, a recycled material, a carbohydrate waste, corn cob, corn stover, wheat straw, nut hulls, soy hulls, switchgrass, gammagrass, paper, or a combination thereof.

### Extraction and Purification of Metabolites

In one aspect, the methods disclosed herein can be used to increase the production of metabolites by cells. In some aspects, the metabolites are secreted into a culture medium and collected. In other aspects, the metabolites remain in the cells, requiring the cells to be lysed prior to collection and purification of the metabolites.

In one aspect, prior to collection of any metabolite(s) of interest, fermentation can be stopped. In some aspects, the micro-current will be withdrawn or turned off (e.g., by turning off a power supply to a biofermenter or a similar mechanism). In another aspect, an enzyme such as, for example, lyticase can optionally be used to lyse cells following fermentation. In still another aspect, the cell culture can optionally be autoclaved for a sufficient time following cell lysis in order to ensure no living cells remain in the culture. Following lysis and autoclaving, or instead of performing these two processes, centrifugation, sonication, and filtration can be performed to facilitate collection of relevant metabolites. In an alternative aspect, culture medium including an increased concentration of the desired metabolite(s) from the biofermenter can be used without further processing.

In any of the above aspects, cell cultures of biological devices such as those disclosed herein, purified metabolites, and/or extracts containing metabolites can be applied to other cells and/or tissues in order to increase metabolite production. In one aspect, the cell cultures, metabolites, and/or extracts are applied to plant tissue such as, for example, plant calluses. Following plant tissue growth, calluses can be crushed and macerated with a solvent in order to extract metabolites from the plant tissue. In one aspect, choice of solvent depends on the chemical characteristics of the metabolite being extracted. For example, lycopene would be extracted with a hydrophobic solvent.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### Aspects

The present disclosure can be described in accordance with the following numbered aspects, which should not be confused with the claims.

Aspect 1. A DNA construct comprising the following genetic components:
(a) a gene that encodes β-amyloid precursor protein;
(b) a gene that encodes microtubule associated protein tau (MAPT);
(c) a gene that encodes adipose triglyceride lipase;
(d) a gene that encodes an acyl-CoA dehydrogenase; and
(e) a gene that encodes an O-linked N-acetylglucosamine transferase (OGlcNAcase).

Aspect 2. The DNA construct of aspect 1, wherein the gene that encodes β-amyloid precursor protein has SEQ ID NO. 1 or at least 70% homology thereto.

Aspect 3. The DNA construct of aspect 1, wherein the gene that encodes MAPT has SEQ ID NO. 2 or at least 70% homology thereto.

Aspect 4. The DNA construct of aspect 1, wherein the gene that encodes adipose triglyceride lipase has SEQ ID NO. 3 or at least 70% homology thereto.

Aspect 5. The DNA construct of aspect 1, wherein the gene that encodes the acyl-CoA dehydrogenase has SEQ ID NO. 4 or at least 70% homology thereto.

Aspect 6. The DNA construct of aspect 1, wherein the gene that encodes the OGlcNAcase has SEQ ID NO. 5 or at least 70% homology thereto.

Aspect 7. The DNA construct of aspect 1, wherein the construct further comprises at least one promoter.

Aspect 8. The DNA construct of aspect 7, wherein the at least one promoter is a T3 promoter, a T7 promoter, an iron promoter, a GAL1 promoter, or any combination thereof.

Aspect 9. The DNA construct of aspect 8, wherein the at least one promoter is a GAL1 promoter, and the GAL1 promoter is positioned before the gene that encodes β-amyloid precursor protein, the gene that encodes MAPT; the gene that encodes adipose triglyceride lipase; the gene that encodes the acyl-CoA dehydrogenase; the gene that encodes the OGlcNAcase, or any combination thereof.

Aspect 10. The DNA construct of aspect 1, wherein the DNA construct further comprises a gene that confers resistance to an antibiotic.

Aspect 11. The DNA construct of aspect 10, wherein the antibiotic comprises tetracycline, neomycin, kanamycin, ampicillin, hygromycin, chloramphenicol, amphotericin B, bacitracin, carbapenem, cephalosporin, ethambutol, fluoroquinolones, isoniazid, methicillin, oxacillin, vancomycin, streptomycin, quinolines, rifampin, rifampicin, sulfonamides, cephalothin, erythromycin, streptomycin, gentamycin, penicillin, other commonly-used antibiotics, or a combination thereof.

Aspect 12. The DNA construct of aspect 1, wherein the DNA construct further comprises at least one terminator.

Aspect 13. The DNA construct of aspect 12, wherein the at least one terminator is a CYC1 terminator.

Aspect 14. The DNA construct of aspect 1, wherein the DNA construct further comprises a reporter protein.

Aspect 15. The DNA construct of aspect 14, wherein the reporter protein is a fluorescent reporter protein.

Aspect 16. The DNA construct of aspect 15, wherein the fluorescent reporter protein is a red fluorescent protein, a cyan fluorescent protein, a green fluorescent protein, or a yellow fluorescent protein.

Aspect 17. The DNA construct of aspect 16, wherein the fluorescent reporter protein is a green fluorescent protein.

Aspect 18. The DNA construct of aspect 17, wherein the green fluorescent protein is SEQ ID NO. 6 or has at least 70% homology thereto.

Aspect 19. The DNA construct of aspect 1, wherein the construct comprises from 5′ to 3′ the following genetic components in the following order: (a) the gene that encodes β-amyloid precursor protein; (b) the gene that encodes MAPT; (c) the gene that encodes adipose triglyceride lipase; (d) the gene that encodes the acyl-CoA dehydrogenase; and (e) a gene that encodes the OGlcNAcase.

Aspect 20. The DNA construct of aspect 1, wherein the construct comprises from 5′ to 3′ the following genetic components in the following order: (a) the gene that encodes β-amyloid precursor protein having SEQ ID NO. 1 or at least 70% homology thereto; (b) the gene that encodes MAPT having SEQ ID NO. 2 or at least 70% homology thereto; (c) the gene that encodes adipose triglyceride lipase having SEQ ID NO. 3 or at least 70% homology thereto; (d) the gene that encodes the acyl-CoA dehydrogenase having SEQ ID NO. 4 or at least 70% homology thereto; and (e) a gene that encodes the OGlcNAcase having SEQ ID NO. 5 or at least 70% homology thereto.

Aspect 21. The DNA construct of aspect 1, wherein the construct comprises from 5′ to 3′ the following genetic components in the following order: (a) the gene that encodes β-amyloid precursor protein; (b) a CYC1 terminator; (c) a GAL1 promoter; (d) the gene that encodes MAPT; (e) a CYC1 terminator; (f) a GAL1 promoter; (g) the gene that encodes adipose triglyceride lipase; (h) a CYC1 terminator; (i) a GAL1 promoter; (j) the gene that encodes the acyl-CoA dehydrogenase; (k) a CYC1 terminator; (I) a GAL1 promoter; and (m) a gene that encodes the OGlcNAcase.

Aspect 22. The DNA construct of aspect 1, wherein the construct comprises from 5′ to 3′ the following genetic components in the following order: (a) the gene that encodes β-amyloid precursor protein having SEQ ID NO. 1 or at least 70% homology thereto; (b) a CYC1 terminator; (c) a GAL1 promoter; (d) the gene that encodes MAPT having SEQ ID NO. 2 or at least 70% homology thereto; (e) a CYC1 terminator; (f) a GAL1 promoter; (g) the gene that encodes adipose triglyceride lipase having SEQ ID NO. 3 or at least 70% homology thereto; (h) a CYC1 terminator; (i) a GAL1 promoter; (j) the gene that encodes the acyl-CoA dehydrogenase having SEQ ID NO. 4 or at least 70% homology thereto; (k) a CYC1 terminator; (I) a GAL1 promoter; and (m) a gene that encodes the OGlcNAcase having SEQ ID NO. 5 or at least 70% homology thereto.

Aspect 23. The DNA construct of aspect 1, wherein the DNA construct has SEQ ID NO. 7.

Aspect 24. A vector comprising the DNA construct of aspect 1.

Aspect 25. The vector of aspect 24, wherein the vector is a plasmid.

Aspect 26. The vector of aspect 25, wherein the plasmid is pWLneo, pSV2cat, pOG44, pXT1, pSG, pSVK3, pBSK, pBSKII, pYES, pYES2, pET, pUC, or pUC19.

Aspect 27. The vector of aspect 26, wherein the vector is pYES2.

Aspect 28. A biological device comprising host cells transformed with the DNA construct in any one of aspects 1-23.

Aspect 29. The device of aspect 28, wherein the host cells comprise fungi or bacteria.

Aspect 30. The device of aspect 29, wherein the fungi comprise Saccharomyces cerevisiae.

Aspect 31. A method for producing a composition for detecting Alzheimer's disease or a concussion, the method comprising growing the biological device of any one of aspects 28-30 for a time sufficient to produce the composition.

Aspect 32. The method of aspect 31, wherein after growing the biological device to produce the composition, the method further comprises the step of lysing the host cells in the composition to produce a lysed composition.

Aspect 33. A composition produced by the method of aspect 31 or 32.

Aspect 34. A method for detecting β-amyloid protein or concussion in a subject, the method comprising admixing the composition of aspect 33 with a biological sample from the subject and detecting a signal from the biological sample.

Aspect 35. The method of aspect 34, wherein the subject is a human.

Aspect 36. The method of aspect 33 or 34, wherein the biological sample comprises blood, serum, plasma, or saliva.

Aspect 37. The method of any one of aspects 34-36, wherein the signal comprises a fluorescence signal.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Preparation of DNA Constructs

The DNA construct was composed of the genetic components described herein and assembled in plasmid vectors (e.g., pYES2, pBAD). Sequences of genes and/or proteins with desired properties were identified in GenBank; these included a gene that encodes β-amyloid precursor protein, a gene that encodes MAPT, a gene that encodes adipose triglyceride lipase, a gene that encodes acyl-CoA dehydrogenase, and a gene that encodes OGlcNAcase. These sequences were synthesized by CloneTex Systems, Inc. (Austin, TX). Other genetic parts were also obtained for inclusion in the DNA constructs including, for example, promoter genes (e.g., GAL1 promoter), reporter genes (e.g., enhanced green fluorescent reporter protein), and terminator sequences (e.g., CYC1 terminator). These genetic parts included restriction sites for ease of insertion into plasmid vectors.

The cloning of the DNA construct into the biological devices was performed as follows. Sequences of individual genes were amplified by polymerase chain reaction using primers that incorporated restriction sites at their 5′ ends to facilitate construction of the full sequence to be inserted into the plasmid. Genes were then ligated using standard protocols to form an insert. The plasmid was then digested with restriction enzymes according to directions and using reagents provided by the enzymes' supplier (Promega). The complete insert, containing restriction sites on each end, was then ligated into the plasmid. Successful construction of the insert and ligation of the insert into the plasmid were confirmed by gel electrophoresis.

In some experiments, each gene was PCR amplified using gene-specific overlap primers and assembled sequences were sub-cloned into a pYES2 vector. PCR amplified pieces of all fragments were combined using homologous recombination technology (Gibson Assembly). Clones obtained after transformation were sequenced and analyzed for DNA sequence accuracy.

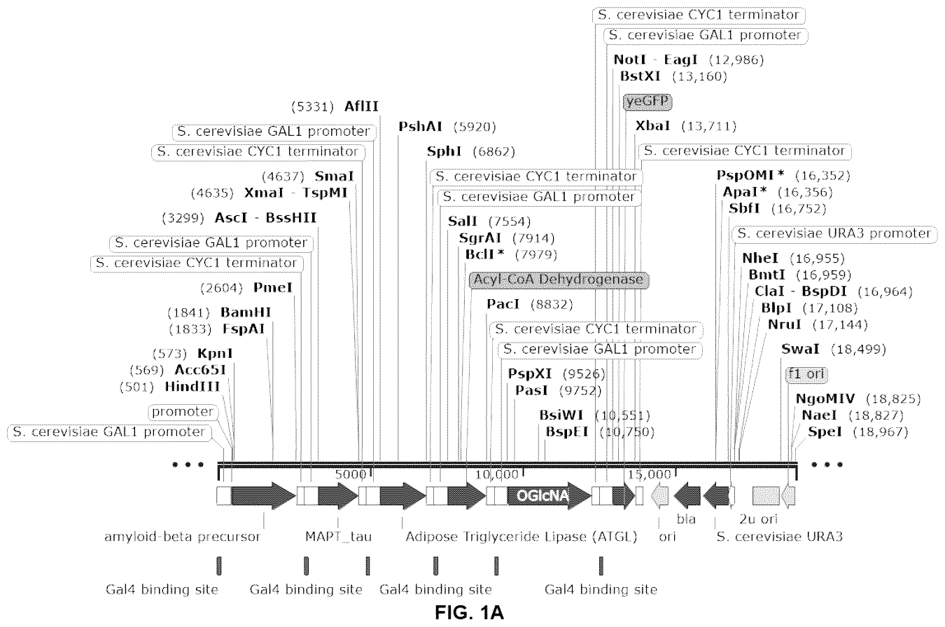

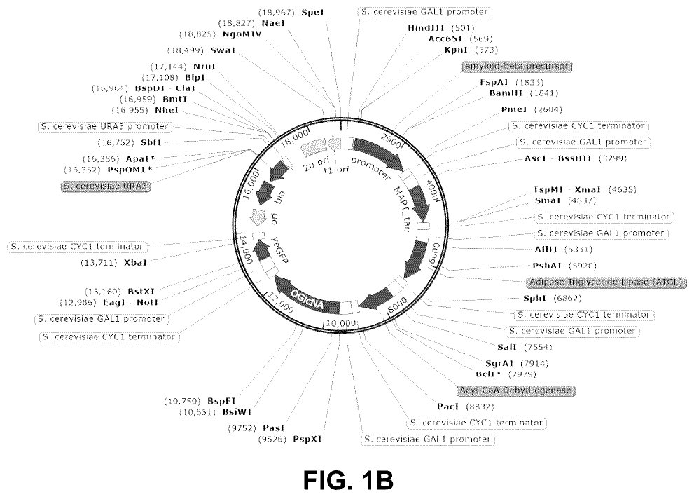

From 5′ to 3′, one version of the construct for producing an Alzheimer's disease and/or concussion-detecting DNA composition or extract includes (a) a gene that encodes β-amyloid precursor protein, (b) a gene that encodes MAPT, (c) a gene that encodes adipose triglyceride lipase, (d) a gene that encodes acyl-CoA dehydrogenase, (e) a gene that encodes OGlcNAcase, and (f) a gene that encodes EGFP (FIGS. 1A-1B).

PCR was used to enhance DNA concentration using a Mastercycler Personal 5332 ThermoCycler (Eppendorf North America) with specific sequence primers and the standard method for amplification (Sambrook, J., E.F. Fritsch, and T. Maniatis, 1989, Molecular Cloning: A Laboratory Manual, 2nd ed., Vol. 1, Cold Spring Harbor Laboratory Press: Cold Spring Harbor, NY). Digestion and ligation were used to ensure assembly of DNA synthesized parts using restriction enzymes and reagents (PCR master mix of restriction enzymes: Xhol, Kpnl, Xbal, EcoRI, BamHI, and Hindlll, with alkaline phosphatase and quick ligation kit, all from Promega). DNA was quantified using a NanoVue spectrophotometer (GE Life Sciences) and a standard UV/Visible spectrophotometer using the ratio of absorbances at 260 nm and 280 nm. In order to verify final ligations, DNA was visualized and purified via electrophoresis using a Thermo EC-150 power supply.

The DNA construct was made with gene parts fundamental for expression of sequences such as, for example, native and constitutive promoters, reporter genes, and transcriptional terminators or stops. Backbone plasmids and synthetic inserts can be mixed together for ligation purposes at different ratios ranging from 1:1, 1:2, 1:3, 1:4, and up to 1:5. In one aspect, the ratio of backbone plasmid to synthetic insert is 1:4. After the vector comprising the DNA construct has been produced, the resulting vector can be incorporated into the host cells using the method described below.

### Example 2: Selection of Microorganisms and Development of Competent Cells

Some constructs were produced using transfected yeasts (Saccharomyces cerevisiae, ATCC® 200892™). Yeast cells were made competent by subjecting them to an electrochemical process adapted from Gietz and Schiestl (Nature Protocols, 2007, 2:35-37). Briefly, a single yeast colony was inoculated into 100 mL YPD (yeast extract peptone dextrose) growth media. Yeast was grown overnight on a shaker at 30° C. to OD600=1.0. (Acceptable results were obtained with OD600 values ranging from 0.6 to 1.8.) Cells were centrifuged at 2000 rpm in a tabletop centrifuge and resuspended in 10 mL TEL buffer (10 mM Tris-HCl, 1 mM EDTA, 0.1 M LiAc, pH=7.5) and shaken vigorously overnight at room temperature. Alternatively, INVSc1 cells were prepared to be competent using a kit from Sigma-Aldrich, Inc. Cells were again centrifuged and resuspended in 1 mL TEL buffer. Cells prepared in this manner could be stored in the refrigerator for up to one month.

Alternatively, bacterial devices were constructed with one of the following strains of cells: Escherichia coli, ONESHOT® Top10 competent cells from Life Technologies™, BL21 (DE3) E. coli from Novagen, Inc., or DH5a™ E. coli from Thermo Fisher Scientific.

### Example 3: Transformation of Microbial Cells

Competent cells were stored in the freezer until needed. Cells were thawed on ice and 100 μL of competent cells in TEL buffer were placed in a sterile 1.5 mL microcentrifuge tube. To this was added 5 μL of a 10 mg/mL solution of salmon sperm DNA (carrier DNA). Transforming DNA was added in various amounts. From 1 to 5 μg was sufficient for plasmids from commercial sources, but more DNA was required when transforming yeast with artificial DNA constructs. 10 μL of the DNA device were added to the microcentrifuge tube containing the competent yeast cells and the contents of the tube were mixed. The DNA-yeast suspension was incubated for 30 min at room temperature.

A PLATE solution (consisting of 40% PEG-3350 in 1 x TEL buffer) was prepared. 0.7 mL of PLATE solution was added to the DNA-yeast suspension and the contents were mixed thoroughly and incubated for 1 h at room temperature. The mixture was placed in an electromagnetic chamber for 30 minutes. Cells were then heated at 42° C. for 5-10 minutes and 250 μL aliquots were plated on yeast malt agar to which selective growth compounds had been added. Plates were incubated overnight at 30° C.

DNA expression and effectiveness of transformation were determined by fluorescence of the transformed cells expressed in fluorescence units (FSUs) using a 20/20 Luminometer (Promega) according to a protocol provided by the manufacturer. Plasmid DNA extraction, purification, PCR, and gel electrophoresis were also used to confirm transformation. Different transformed devices were obtained. Different types of fluorescent reporter proteins were used (e.g., yellow, red, green, and cyan) for all transformed cells and/or constructs. However, the yellow fluorescent protein was preferred. When no fluorescent reporter protein was assembled, no fluorescence was observed.

S. cerevisiae cells were subjected to transformation with the modified pYES2 plasmids for producing metal- and contaminant-binding components as described above. Transformed yeast cells were incubated for 30 min at 28-30° C. Colonies of transformed yeast cells were selected, their DNA isolated and subjected to PCR amplification. Two control treatments were also carried out: (1) a negative control involving competent yeast and nuclease free water instead of a plasmid and (2) a positive control involving competent yeast with unmodified pYES2 plasmid.

Four clones were selected from a transformed plate and processed for full-length DNA sequencing. A clone with 100% DNA sequence accuracy was selected for further processing and was used to obtain a high concentration of plasmid construct at a mid-scale plasmid purification level. Yeast competent cells were transformed with the recombinant plasmid and selected on synthetic complete (SC) dropout plate deficient in uracil. Well isolated clones were isolated and preserved in YPD medium containing 15% glycerol for storage at −80° C.

### Example 4: Production of Microbial Extracts and Metabolites

Microbial Extracts Containing Alzheimer's Disease and/or Concussion Detecting Metabolites

The following non-limiting procedure was used to produce the disclosed extracts: Method
(a) Yeasts transformed with the device depicted in FIGS. 1A-1B were fermented at 37° C. for 48 hours, where culture was conducted with 25 mL of device inoculum in 1 L Luria broth and having 1 μg/mL ampicillin and 100 μM isopropyl-β-D-thiogalactopyranoside (IPTG).

(b) The culture was sterilized by autoclaving at 121° C. for 30 minutes and then centrifuged.

(c) The mixture was filtered with an 0.45 μm filter to produce a supernatant composed of the desired extract.

### Example 5: Detection of Alzheimer's Disease Using the Devices

### Materials and Methods

Lysates and extracts were produced as follows. A culture of a disclosed device was removed from a −80° C. freezer and activated in yeast malt broth overnight at 30° C. and shaking at 115 rpm. Approximately 1 mL device culture was added to 9 mL yeast malt broth. Following overnight growth, these inoculums were added to 200 mL yeast malt broth and incubated again at 30° C. for 72 hours. Glucosamine at 1 mg/mL, raffinose at 2% (w/v), and galactose at 1% (w/v) were added after 4 hours of incubation.

These cultures were centrifuged at 9000 rpm for 10 min and the pellets were resuspended in ice cold PBS (with 1 mL of PBS for every 10 ml of culture) and centrifuged at maximum rpm for 2 min. This PBS wash was repeated 2 times. Following the PBS wash, the pellet was resuspended in sterile deionized water at 20 mL per gram of pellet. Lyticase was added at 2.5 μg/mL and the solution was incubated for 1 h at 37° C. This solution was centrifuged at 9000 rpm for 10 min and filtered using a 0.45 μm nitrocellulose membrane.

The lysate/extract thus produced was mixed with patient saliva in a proportion of 3:1 (3 parts lysate, 1 part saliva). The mixtures were incubated for 30 min at 37° C. and shaking at 150 rpm.

Fluorescence measurements were made in a GloMax Multi-detection System with Instinct™ Software: Base Instrument with Shaking (PROMEGA, Madison, Wisconsin USA) 5 different modules were used (UV, AFC, Blue, Green, and Red) with the Blue module providing the clearest results. The volume used per sample for measurement was 100 μL per well (a plate contains 96 wells). Mean and standard deviation were calculated and ANOVA was conducted.

### Conjugation Protocol

For verification of results, a modified sulfo-cyanine antibody labeling kit (from Lumiprobe) was used. The protocol for labeling was as follows: The solution pH was adjusted to 8. To activate the fluorophore (sulfo-Cyanine3 NHS ester) 10 μL of DMSO were added to microcentrifuge tubes with lyophilized fluorophore. A mixture of the Lysate-Saliva with sodium bicarbonate solution (30 μl and 70 μL, respectively) was prepared and then this was added to the fluorophore and incubated overnight at room temperature. The purification column was prepared per manufacturer directions. The fluorophore/lysate mixture was centrifuged for 2 min at 3800 rpm and 400 μL PBS buffer was added, followed by centrifugation for 2 min at 3800 rpm. The labeled antibody was purified in the column and collected fractions were centrifuged for 2 min at 3800 rpm. The samples were measured in GloMax®-Multi+ Detection System as described above. Results are presented in Table 6 below:

TABLE 6
Fluorescence Results for Human Subjects

| | | | | |
|---|---|---|---|---|
| Gender | Age | Fluorescence | Medical Diagnosis | MRI |

Alzheimer's patients

| | | | | |
|---|---|---|---|---|
| Female | 92 | 17,119 | Mental Confusion | Bilateral moderate front parietal |
| | | | and Memory Loss | atrophy, Moderately severe |
| | | | | atrophy of the Hippocampi and |
| | | | | Bilateral aphasia |
| Female | 93 | 17,893 | Disorientation, | Global Cortical Atrophy and |
| | | | Mental Confusion | Atrophy in the mesial region |
| | | | and Memory Loss | of the temporal lobes |
| Female | 78 | 16,935 | Memory Loss | Mild bilateral front parietal |
| | | | | atrophy and Mild supratentorial |
| | | | | hydrocephalus |

Pre-Alzheimer's patients

| | | | |
|---|---|---|---|
| Female | 15,642 | In remission from | Non- MRI |
| | | leukemia | |
| Female | 15,337 | In remission from | Non-MRI |
| | | breast cancer and | |
| | | family with Alzheimer | |
| | | diagnosis | |

Healthy

| | | | | |
|---|---|---|---|---|
| Female | 52 | 15,281 | Healthy | Mild bilateral mesial |
| | | | | temporal atrophy |
| Male | 30 | 14,574 | Healthy | Without Alteration |

The results show good correlation between fluorescence detection using the disclosed devices and medical/clinical MRI diagnosis. The patients with higher fluorescence above 15,000 RFU show atrophy in the MRI results as well corresponding clinical/medical diagnosis, which include memory loss, mental confusion, and/or disorientation. Likewise, the pre-Alzheimer's patient show good correlation between fluorescence as clinical/medical diagnosis and MRI. Furthermore, the healthy patients show the lowest fluorescence values (less than 15,000 RFU), and no indication of atrophy according to MRI as well as having no clinical/medical deficiencies.

In some aspects, the tests described use only saliva and are thus fast, cost-effective, and non-invasive, as opposed to blood tests or MRI. In an alternative aspect, the disclosed extracts can be used to detect β-amyloid protein and/or the effects of concussion in serum, blood, or plasma, in addition to saliva.

These results also encourage to carry out not only more tests for Alzheimer's but to do tests for identifying beta-amyloid in concussion patients, as it is one of the objectives of the denoted technology. Throughout this application, various publications are referenced. The disclosures of these publications in their entireties are hereby incorporated by reference into this application in order to more fully describe the compounds, compositions, and methods described herein.

Various modifications and variations can be made to the compounds, compositions, and methods described herein. Other aspects of the compounds, compositions, and methods described herein will be apparent from consideration of the specification and practice of the compounds, compositions, and methods disclosed herein. It is intended that the specification and examples be exemplary.

What is claimed is:

1. A DNA construct comprising the following genetic components:
(a) a gene that encodes β-amyloid precursor protein;
(b) a gene that encodes microtubule associated protein tau (MAPT);
(c) a gene that encodes adipose triglyceride lipase;
(d) a gene that encodes an acyl-CoA dehydrogenase; and
(e) a gene that encodes an O-linked N-acetylglucosamine transferase (OGlcNAcase).

2. The DNA construct of claim 1, wherein the gene that encodes β-amyloid precursor protein has SEQ ID NO. 1 or at least 70% homology thereto.

3. The DNA construct of claim 1, wherein the gene that encodes MAPT has SEQ ID NO. 2 or at least 70% homology thereto.

4. The DNA construct of claim 1, wherein the gene that encodes adipose triglyceride lipase has SEQ ID NO. 3 or at least 70% homology thereto.

5. The DNA construct of claim 1, wherein the gene that encodes the acyl-CoA dehydrogenase has SEQ ID NO. 4 or at least 70% homology thereto.

6. The DNA construct of claim 1, wherein the gene that encodes the OGlcNAcase has SEQ ID NO. 5 or at least 70% homology thereto.

7. The DNA construct of claim 1, wherein the construct further comprises at least one promoter.

8. The DNA construct of claim 7, wherein the at least one promoter is a T3 promoter, a T7 promoter, an iron promoter, a GAL1 promoter, or any combination thereof.

9. The DNA construct of claim 8, wherein the at least one promoter is a GAL1 promoter, and the GAL1 promoter is positioned before the gene that encodes β-amyloid precursor protein, the gene that encodes MAPT; the gene that encodes adipose triglyceride lipase;
the gene that encodes the acyl-CoA dehydrogenase; the gene that encodes the OGlcNAcase, or any combination thereof.

10. The DNA construct of claim 1, wherein the DNA construct further comprises at least one terminator.

11. The DNA construct of claim 10, wherein the at least one terminator is a CYC1 terminator.

12. The DNA construct of claim 1, wherein the DNA construct further comprises a fluorescent reporter protein.

13. The DNA construct of claim 12, wherein the fluorescent reporter protein is a red fluorescent protein, a cyan fluorescent protein, a green fluorescent protein, or a yellow fluorescent protein.

14. The DNA construct of claim 13, wherein the fluorescent reporter protein is a green fluorescent protein.

15. The DNA construct of claim 14, wherein the green fluorescent protein is SEQ ID NO. 6 or has at least 70% homology thereto.

16. The DNA construct of claim 1, wherein the construct comprises from 5′ to 3′ the following genetic components in the following order: (a) the gene that encodes β-amyloid precursor protein; (b) the gene that encodes MAPT; (c) the gene that encodes adipose triglyceride lipase; (d) the gene that encodes the acyl-CoA dehydrogenase; and (e) a gene that encodes the OGlcNAcase.

17. The DNA construct of claim 1, wherein the construct comprises from 5′ to 3′ the following genetic components in the following order: (a) the gene that encodes β-amyloid precursor protein having SEQ ID NO. 1 or at least 70% homology thereto; (b) the gene that encodes MAPT having SEQ ID NO. 2 or at least 70% homology thereto; (c) the gene that encodes adipose triglyceride lipase having SEQ ID NO. 3 or at least 70% homology thereto; (d) the gene that encodes the acyl-CoA dehydrogenase having SEQ ID NO. 4 or at least 70% homology thereto; and (e) a gene that encodes the OGlcNAcase having SEQ ID NO. 5 or at least 70% homology thereto.

18. The DNA construct of claim 1, wherein the construct comprises from 5′ to 3′ the following genetic components in the following order: (a) the gene that encodes β-amyloid precursor protein; (b) a CYC1 terminator; (c) a GAL1 promoter; (d) the gene that encodes MAPT; (e) a CYC1 terminator; (f) a GAL1 promoter; (g) the gene that encodes adipose triglyceride lipase; (h) a CYC1 terminator; (i) a GAL1 promoter; (j) the gene that encodes the acyl-CoA dehydrogenase; (k) a CYC1 terminator; (I) a GAL1 promoter; and (m) a gene that encodes the OGlcNAcase.

19. The DNA construct of claim 1, wherein the construct comprises from 5′ to 3′ the following genetic components in the following order: (a) the gene that encodes β-amyloid precursor protein having SEQ ID NO. 1 or at least 70% homology thereto; (b) a CYC1 terminator; (c) a GAL1 promoter; (d) the gene that encodes MAPT having SEQ ID NO. 2 or at least 70% homology thereto; (e) a CYC1 terminator; (f) a GAL1 promoter; (g) the gene that encodes adipose triglyceride lipase having SEQ ID NO. 3 or at least 70% homology thereto; (h) a CYC1 terminator; (i) a GAL1 promoter; (j) the gene that encodes the acyl-CoA dehydrogenase having SEQ ID NO. 4 or at least 70% homology thereto; (k) a CYC1 terminator; (I) a GAL1 promoter; and (m) a gene that encodes the OGlcNAcase having SEQ ID NO. 5 or at least 70% homology thereto.

20. The DNA construct of claim 1, wherein the DNA construct has SEQ ID NO. 7.

21. A vector comprising the DNA construct of claim 1.

22. The vector of claim 21, wherein the vector is a plasmid.

23. The vector of claim 22, wherein the plasmid is pWLneo, pSV2cat, pOG44, pXT1, PSG, pSVK3, pBSK, pBSKII, pYES, pYES2, pET, pUC, or pUC19.

24. The vector of claim 23, wherein the vector is pYES2.

25. A biological device comprising host cells transformed with the DNA construct of claim 1.

26. The device of claim 25, wherein the host cells comprise fungi or bacteria.

27. The device of claim 26, wherein the fungi comprise Saccharomyces cerevisiae.

28. A method for producing a composition for detecting Alzheimer's disease or a concussion, the method comprising growing the biological device of claim 25 for a time sufficient to produce the composition.

29. The method of claim 28, wherein after growing the biological device to produce the composition, the method further comprises the step of lysing the host cells in the composition to produce a lysed composition.

30. A composition produced by the method of claim 28.
